(19) United States
(12) Patent Application Publication
(10) Pub. No.: US 2002/0009751 A1
(43) Pub. Date: Jan. 24, 2002

(54) Drosophila homologues of genes and proteins implicated in metabolism and methods of use

(76) Inventors: Cindy Seidel-Dugan, Benicia, CA (US); Lori Friedman, San Francisco, CA (US); Justin Torpey, San Francisco, CA (US); Kevin Patrick Keegan, San Leandro, CA (US); Jonathan C. Heller, San Francisco, CA (US); Thomas J. Stout, San Francisco, CA (US)

Correspondence Address: JAN P. BRUNELLE, EXELIXIS, INC., 170 HARBOR WAY, P.O. BOX 511, SOUTH SAN FRANCISCO, CA, 94083-0511, US

(21) Appl. No.: 09/740,046

(22) Filed: Dec. 18, 2000

Related U.S. Application Data
(60) Provisional application No. 60/172,484, filed on Dec. 17, 1999.
(60) Provisional application No. 60/172,482, filed on Dec. 17, 1999.
(60) Provisional application No. 60/178,411, filed on Jan. 27, 2000.
(60) Provisional application No. 60/191,881, filed on Mar. 23, 2000.
(60) Provisional application No. 60/192,142, filed on Mar. 23, 2000.

(51) Int. Cl. G01N 33/53; C07H 21/04; C12N 9/00; C12P 21/02; C12N 5/06

(52) U.S. Cl. 435/7.1; 435/325; 435/183; 435/69.1; 536/23.2

(57) ABSTRACT

Novel nucleic acids that are homologs of genes implicated in metabolism and that have been isolated from Drosophila melanogaster are described. These nucleic acids and proteins can be used to genetically modify metazoan invertebrate organisms, such as insects and worms, or cultured cells, resulting in novel gene expression or mis-expression. The genetically modified organisms or cells can be used in screening assays to identify candidate compounds which are potential therapeutics that interact with gene products implicated in metabolism. They can also be used in methods for studying gene activity and identifying other genes that modulate the function of, or interact with, genes implicated in metabolism.

### REFERENCE TO PENDING APPLICATION

This application claims priority to provisional applications 60/172,484, filed on Dec. 17, 1999; 60/172,482, filed on Dec. 17, 1999; 60/178,411, filed on Jan. 27, 2000; 60/191,881, filed on Mar. 23, 2000; and 60/192,142, filed on Mar. 23, 2000; the entire contents of which are incorporated herein by reference.

### BACKGROUND OF THE INVENTION

There is much interest within the pharmaceutical industry to understand the mechanisms involved in metabolism, particularly on the molecular level, so that drugs can be developed for the treatment or prevention of metabolic diseases.

APS protein (adapter protein with pleckstrin homology (PH) and src homology-2 (SH2) domain) is the newest member of a family of tyrosine kinase adapter proteins including SH2-B and Lnk. Both SH2-B and APS are tyrosine phosphorylated by the insulin receptor upon activation by insulin (Moodie et al., J. Biol. Chem. (1999) 274(16): 11186-11193; Kotani et al., Biochem. J. (1998) 335:103-109). These molecules are involved in tyrosine kinase signaling. APS interacts with the insulin receptor kinase activation loop through its SH2 domain, and insulin stimulates the tyrosine-phosphorylation of APS by the insulin receptor (Ahmed et al., Biochem. J. (1999) 341(pt 3):665-668). This suggests a potential role for APS in insulin-regulated metabolic signaling pathways. APS inhibits PDGF-induced mitogenesis (Yokouchi et al., Oncogene (1999) 18(3):759-67). Due to its role in both insulin signaling and mitogenesis, this gene may be valuable as a therapeutic target.

Cytochrome P450s (Nebert D. W., and Gonzalez F. J., Annu. Rev. Biochem. 56:945-993(1987); Coon M. J., et al., FASEB J. 6:669-673(1992); Guengerich F. P., J. Biol. Chem. 266:10019-10022(1991)) are a group of enzymes involved in the oxidative metabolism of a high number of natural compounds (such as steroids, fatty acids, prostaglandins, leukotrienes, etc) as well as drugs, carcinogens and mutagens. Based on sequence similarities, P450s have been classified into about forty different families (Nelson D. R., et al., DNA Cell Biol. 12:1-51(1993); Degtyarenko K. N., and Archakov A. I., FEBS Lett. 332:1-8(1993)). P450s are heme proteins of 400 to 530 amino acids. A conserved cysteine residue in the C-terminal part of P450s is involved in binding the heme iron in the fifth coordination site. The CYP4 family of p450s is involved in CYP-mediated processes such as xenobiotic detoxification and the biosynthesis of steroid molecules involved in the regulation of a variety of biological processes. Evidence exists to suggest that CYP4 family members in invertebrates may play a role similar to their counterpart mammalian CYP4 isozymes: steroid biosynthesis. Biosynthesis of ecdysteroids, arthropod steroid molting hormones, proceeds from dietary cholesterol through a complex pathway known to involve CYPs (Dauphin-Villemant C, et al., Biochem. Biophys. Res. Commun. Oct. 22, 1999; 264(2):413-8). Recently a novel cytochrome P450 (CYP4C15) was cloned from an arthropod which is differentially expressed in the steroidogenic glands (Dauphin-Villemant C, et al., Biochem. Biophys. Res. Commun. Oct. 22, 1999; 264(2):413-8). Northern blots demonstrated predominant expression of this gene in the active molting glands, suggesting a role in ecdysteroid biosynthesis rather than detoxification (Dauphin-Villemant C, et al., Biochem. Biophys. Res. Commun. Oct. 22, 1999; 264(2):413-8). This is an example of the myriad of biological processes that are regulated or mediated by steriod hormones. CYPs are inolved in the biosynthesis of virtually all of these hormones because of their ability to hydroxylate unactivated hydrocarbons. CYPs are therefore attractive as compound targets because molecules could be designed to inhibit CYPs that would interfere with the production of essential steroids and fatty acids.

Insulin-like growth factors (IGF) I and II are chemically-related single-chain peptides with significant homologies to insulin and to other members of the insulin family of growth factors. Mammalian IGFs (IGF-I and IGF-II) are essential for normal growth and development. Their functions include mediation of growth hormone action, stimulation of growth of cultured cells, stimulation of the action of insulin, and involvement in development and growth. Their actions are mediated primarily by their interactions with the type IIGF receptor (IGF-I receptor), a transmembrane tyrosine kinase. The ligands and the IGF-I receptor are structurally related to insulin and to the insulin receptor, respectively (LeRoith D, Kavsan V M, Koval A P, Roberts C T Jr Mol Reprod Dev (1993) 4:332-8; Rotwein P; Growth Factors 1991;5(1):3-18). Insulin-Like Growth Factor-I (IGF-I, originally called somatomedin C) is a growth factor structurally related to insulin. IGF-I is the primary protein involved in responses of cells to growth hormone (GH): that is, IGF-I is produced in response to GH and then induces subsequent cellular activities, particularly on bone growth. It is the activity of IGF-I in response to GH that gave rise to the term somatomedin. Insulin-Like Growth Factor-II is almost exclusively expressed in embryonic and neonatal tissues. Following birth, the level of detectable IGF-II protein falls significantly. For this reason IGF-II is thought to be a fetal growth factor (DeChiara T M, Efstratiadis A, Robertson E J. Nature (1990) 345:78-80).

The enzymes that terminate the signal transduction processes and regulate the levels of soluble inositol phosphate and phospholipid messengers are essential for proper cell function. Distinct isoforms of 5-polyphosphates may play specific roles in inositol phosphate and phosphatidylinositol metabolism (Drayer et al., Biochem. Soc. Trans. (1996) 24:1001-1005; Berridge et al., Nature (1993) 361:315-325). The structural features that classify src homology 2-containing inositol 5′-phosphatase (SHIP) are an amino terminal src homology 2 (SH2) domain, a central 5′-phosphotidylinositol phosphatase activity domain, a phospho-tyrosine binding (PTB) consensus sequence, and a proline-rich region at the carboxyl tail (Ishihara, et al., Biochem. Biophys. Res. Comm. (1999) 260:265-272), which is potentially an SH3 binding domain (Wisniewski, et al., Blood 1999, 93(8):2707-2720). Two isozymes have been characterized in rat and human, designated as SHIP1 and SHIP2. SHIP1 is present in hematopoietic cells and human SHP2 is present in the heart and skeletal muscle, which are key target tissues of insulin action (Pesesse et al., Biochem. Biophys. Res. Comm. (1997) 239:697-700). Slight structural features distinguish SHIP1 and SHIP2. Both rat and human SHIP2 have only one C-terminal PTB binding consensus sequence (NPAY), while rat SHIP1 has two C-terminal PTB sites (NPNY, NPLY) (Ishihara et al, supra). SHIP2 expressed in E. coli has 5′-phosphatase activity (Pesesse, supra). One of its substrates, phosphatidylinositol 3,4,5-trisphosphate, is thought to be a second messenger of phosphatidyl-inositol 3′-kinase (PI3-kinase) mediated signaling in response to growth factors and insulin (Habib et al., J. Biol. Chem. (1998) 273(29):18605-18609; Guilherme, et al., J. Biol. Chem. (1996) 271(47):29533-29536). This pathway is implicated in mitogenesis, oncogenic transformation, and apoptosis. SHIP2 also appears to negatively regulate PI3-kinase downstream products produced by insulin signaling (Ishihara et al., supra). The SH2 domain of SHIP2 has been shown to interact with Shc at its phosphorylated Y317 residue (Ishihara, supra; Wada, T., et al., Endocrinology 1999, 140(10): 4585-4594). Phosphorylated Shc binds to Grb2, via its SH2 domain, which is important for Ras-MAP kinase activation (Ishihara et al., supra; Wada, supra). Evidence suggests that a competitive interaction between SHIP2 and Shc may reduce Ras activity resulting in negative regulation of mitogenesis (Ishihara et al., supra; Wada, supra). Furthermore, it has been demonstrated that the SH2 domain of SH[P plays a critical part in its negative regulatory role in insulin-induced mitogenesis (Wada, supra).

### SUMMARY OF THE INVENTION

It is an object of the present invention to provide invertebrate homologs of genes implicated in metabolism that can be used in genetic screening methods to characterize pathways that metabolism-related genes may be involved in as well as other interacting genetic pathways. It is also an object of the invention to provide methods for screening compounds that interact with metabolism-related genes such as those that may have utility as therapeutics. These and other objects are provided by the present invention which concerns the identification and characterization of novel genes in Drosophila melanogaster. Isolated nucleic acid molecules are provided that comprise nucleic acid sequences encoding homologs of the following metabolism-related genes: APS, hereinafter referred to as dmAPS; cytochrome P450, hereinafter referred to as dmCYP; IGF II, hereinafter referred to as dmIGF; and SHIP2, hereinafter referred to as dmSHIP2A and dmSHIP2B.

The invention also includes novel fragments and derivatives of these nucleic acid molecules. Vectors and host cells comprising the subject nucleic acid molecules are also described, as well as metazoan invertebrate organisms (e.g. insects, coelomates and pseudocoelomates) that are genetically modified to express or mis-express subject proteins.

An important utility of the novel subject nucleic acids and proteins is that they can be used in screening assays to identify candidate compounds that are potential therapeutics that interact with subject proteins. Such assays typically comprise contacting a subject protein or fragment with one or more candidate molecules, and detecting any interaction between the candidate compound and the subject protein. The assays may comprise adding the candidate molecules to cultures of cells genetically engineered to express subject proteins, or alternatively, administering the candidate compound to a metazoan invertebrate organism genetically engineered to express subject protein.

The genetically engineered metazoan invertebrate animals of the invention can also be used in methods for studying subject gene activity. These methods typically involve detecting the phenotype caused by the expression or mis-expression of the subject protein. The methods may additionally comprise observing a second animal that has the same genetic modification as the first animal and, additionally has a mutation in a gene of interest. Any difference between the phenotypes of the two animals identifies the gene of interest as capable of modifying the function of the gene encoding the subject protein.

### DETAILED DESCRIPTION OF THE INVENTION

The use of invertebrate model organism genetics and related technologies can greatly facilitate the elucidation of biological pathways (Scangos, Nat. Biotechnol. (1997) 15:1220-1221; Margolis and Duyk, supra). Of particular use is the insect model organism, Drosophila melanogaster (hereinafter referred to generally as “Drosophila”). An extensive search for homologues of vertebrate metabolism nucleic acids and their encoded proteins in Drosophila was conducted in an attempt to identify new and useful tools for probing the function and regulation of such genes, and for use as targets in drug discovery.

The novel nucleic acids encoded proteins that are homologs of the following human proteins implicated in metabolism: APS, cytochrome p450, IGFII, and SHIP2. The nucleic acids and proteins of the invention are collectively referred to as “subject nucleic acids”, “subject genes”, or “subject proteins”. The newly identified subject nucleic acids can be used for the generation of mutant phenotypes in animal models or in living cells that can be used to study regulation of subject genes, and subject proteins can be used as drug targets. Due to the ability to rapidly carry out large-scale, systematic genetic screens, the use of invertebrate model organisms such as Drosophila has great utility for analyzing the expression and mis-expression of subject proteins. Thus, the invention provides a superior approach for identifying other components involved in the synthesis, activity, and regulation of subject proteins. Systematic genetic analysis of subject genes using invertebrate model organisms can lead to the identification and validation of compound targets directed to components of the subject pathway. Model organisms or cultured cells that have been genetically engineered to express subject genes can be used to screen candidate compounds for their ability to modulate subject genes' expression or activity, and thus are useful in the identification of new drug targets, therapeutic agents, diagnostics and prognostics useful in the treatment of metabolic disorders.

The details of the conditions used for the identification and/or isolation of novel subject nucleic acids and proteins are described in the Examples section below. Various non-limiting embodiments of the invention, applications and uses of these novel subject genes and proteins are discussed in the following sections. The entire contents of all references, including patent applications, cited herein are incorporated by reference in their entireties for all purposes. Additionally, the citation of a reference in the preceding background section is not an admission of prior art against the claims appended hereto.

### Nucleic Acids of the Invention

The invention relates generally to nucleic acid sequences of APS, cytochrome P450, IGF2, and SHIP2, and more particularly these nucleic acid sequences of Drosophila (dmAPS, dmCYP, dmIGF, and dmSHIP2A and dmSHIP2B), and methods of using these sequences. The invention provides nucleic, nucleic acid sequences that were isolated from Drosophila and encode homologs of APS (dmAPS; SEQ ID NO:1), cytochrome P450 (dmCYP; SEQ ID NO: 3), IGF2 (dmIGF; SEQ ID NO: 5), and SHIP (dmSHIP2A and dmSHIP2B; SEQ ID NOs: 7 and 9, respectively), as described in the Examples below. In addition to the fragments and derivatives of SEQ ID NOs:1, 3, 5, 7, and 9 as described in detail below, the invention includes the reverse complements thereof. Also, the subject nucleic acid sequences, derivatives and fragments thereof may be RNA molecules comprising the nucleotide sequence of SEQ ID NOs:1, 3, 5, 7, and 9 (or derivatives or fragments thereof) wherein the base U (uracil) is substituted for the base T (thymine). The DNA and RNA sequences of the invention can be single- or double-stranded. Thus, the term “isolated nucleic acid sequence”, as used herein, includes the reverse complement, RNA equivalent, DNA or RNA single- or double-stranded sequences, and DNA/RNA hybrids of the sequence being described, unless otherwise indicated.

Fragments of the subject nucleic acid sequences can be used for a variety of purposes. Interfering RNA (RNAi) fragments, particularly double-stranded (ds) RNAi, can be used to generate loss-of-function phenotypes. Subject nucleic acid fragments are also useful as nucleic acid hybridization probes and replication/amplification primers. Certain “antisense” fragments, i.e. that are reverse complements of portions of the coding sequence of SEQ ID NOs:1, 3, 5, 7 and 9 and have utility in inhibiting the function of subject proteins. The fragments are of lengths sufficient to specifically hybridize with the corresponding SEQ ID NOs:1, 3, 5, 7, and 9. The fragments consist of or comprise at least 12, preferably at least 24, more preferably at least 36, and more preferably at least 96 contiguous nucleotides of SEQ ID NOs:1, 3, 5, 7, and 9. When the fragments are flanked by other nucleic acid sequences, the total length of the combined nucleic acid sequence is less than 15 kb, preferably less than 10 kb or less than 5 kb, more preferably less than 2 kb, and in some cases, preferably less than 500 bases.

Additional preferred fragments of SEQ ID NO:1 encode a pleckstrin homology domain, and an SH2 domain, which are located at approximately nucleotides 1301-1367, and 1772-2003, respectively.

Additional preferred fragments of SEQ ID NO:3 encode extracellular or intracellular domains which are located at approximately nucleotides 73-1569.

An additional preferred fragment of SEQ ID NO:5 encodes an insulin family signature which is located at approximately nucleotides 429-473.

An additional preferred fragment of SEQ ID NO:7 comprises approximately nucleotides 285-1239 which encodes the region located between the two transmembrane domains.

Additional preferred fragments of SEQ ID NO:9 encode extracellular or intracellular domains, which are located at approximately nucleotides 214-439, and 490-3554.

The subject nucleic acid sequences may consist solely of SEQ ID NOs:1, 3, 5, 7, and 9 or fragments thereof. Alternatively, the subject nucleic acid sequences and fragments thereof may be joined to other components such as labels, peptides, agents that facilitate transport across cell membranes, hybridization-triggered cleavage agents or intercalating agents. The subject nucleic acid sequences and fragments thereof may also be joined to other nucleic acid sequences (i.e. they may comprise part of larger sequences) and are of synthetic/non-natural sequences and/or are isolated and/or are purified, i.e. unaccompanied by at least some of the material with which it is associated in its natural state. Preferably, the isolated nucleic acids constitute at least about 0.5%, and more preferably at least about 5% by weight of the total nucleic acid present in a given fraction, and are preferably recombinant, meaning that they comprise a non-natural sequence or a natural sequence joined to nucleotide(s) other than that which it is joined to on a natural chromosome.

Derivative subject nucleic acid sequences include sequences that hybridize to the nucleic acid sequence of SEQ ID NOs:1, 3, 5, 7, or 9 under stringency conditions such that the hybridizing derivative nucleic acids are related to the subject nucleic acids by a certain degree of sequence identity. A nucleic acid molecule is “hybridizable” to another nucleic acid molecule, such as a cDNA, genomic DNA, or RNA, when a single stranded form of the nucleic acid molecule can anneal to the other nucleic acid molecule. Stringency of hybridization refers to conditions under which nucleic acids are hybridizable. The degree of stringency can be controlled by temperature, ionic strength, pH, and the presence of denaturing agents such as formamide during hybridization and washing. As used herein, the term “stringent hybridization conditions” are those normally used by one of skill in the art to establish at least a 90% sequence identity between complementary pieces of DNA or DNA and RNA. “Moderately stringent hybridization conditions” are used to find derivatives having at least 70% sequence identity. Finally, “low-stringency hybridization conditions” are used to isolate derivative nucleic acid molecules that share at least about 50% sequence identity with the subject nucleic acid sequence.

The ultimate hybridization stringency reflects both the actual hybridization conditions as well as the washing conditions following the hybridization, and it is well known in the art how to vary the conditions to obtain the desired result. Conditions routinely used are set out in readily available procedure texts (e.g., Current Protocol in Molecular Biology, Vol. 1, Chap. 2.10, John Wiley & Sons, Publishers (1994); Sambrook et al., Molecular Cloning, Cold Spring Harbor (1989)). A preferred derivative nucleic acid is capable of hybridizing to SEQ ID NO:1, 3, 5, 7 or 9 under stringent hybridization conditions that comprise: prehybridization of filters containing nucleic acid for 8 hours to overnight at 65° C. in a solution comprising 6×single strength citrate (SSC) (133 SSC is 0.15 M NaCl, 0.015 M Na citrate; pH 7.0), 5×Denhardt's solution, 0.05% sodium pyrophosphate and 100 μg/ml herring sperm DNA; hybridization for 18-20 hours at 65° C. in a solution containing 6×SSC, 1×Denhardt's solution, 100 μg/ml yeast tRNA and 0.05% sodium pyrophosphate; and washing of filters at 65° C. for 1 h in a solution containing 0.2×SSC and 0.1% SDS (sodium dodecyl sulfate).

Derivative nucleic acid sequences that have at least about 70% sequence identity with SEQ ID NOs:1, 3, 5, 7, or 9 are capable of hybridizing to SEQ ID NOs:1, 3, 5, 7, or 9 under moderately stringent conditions that comprise: pretreatment of filters containing nucleic acid for 6 h at 40° C. in a solution containing 35% formamide, 5×SSC, 50 mM Tris-HCl (pH7.5), 5 mM EDTA, 0.1% PVP, 0.1% Ficoll, 1% BSA, and 500 μg/ml denatured salmon sperm DNA; hybridization for 18-20 h at 40° C. in a solution containing 35% formamide, 5×SSC, 50 mM Tris-HCl (pH7.5), 5 mM EDTA, 0.02% PVP, 0.02% Ficoll, 0.2% BSA, 100 μg/ml salmon sperm DNA, and 10% (wt/vol) dextran sulfate; followed by washing twice for 1 hour at 55° C. in a solution containing 2×SSC and 0.1% SDS.

Other preferred derivative nucleic acid sequences are capable of hybridizing to SEQ ID NOs: 1, 3, 5, 7, or 9 under low stringency conditions that comprise: incubation for 8 hours to overnight at 37° C. in a solution comprising 20% formamide, 5×SSC, 50 mM sodium phosphate (pH 7.6), 5×Denhardt's solution, 10% dextran sulfate, and 20 μg/ml denatured sheared salmon sperm DNA; hybridization in the same buffer for 18 to 20 hours; and washing of filters in 1×SSC at about 37° C. for 1 hour.

As used herein, “percent (%) nucleic acid sequence identity” with respect to a subject sequence, or a specified portion of a subject sequence, is defined as the percentage of nucleotides in the candidate derivative nucleic acid sequence identical with the nucleotides in the subject sequence (or specified portion thereof), after aligning the sequences and introducing gaps, if necessary to achieve the maximum percent sequence identity, as generated by the program WU-BLAST-2.0a19 (Altschul et al., J. Mol. Biol. (1997) 215:403-410; http://blast.wustl.edu/blast/README.html; hereinafter referred to generally as “BLAST”) with all the search parameters set to default values. The HSP S and HSP S2 parameters are dynamic values and are established by the program itself depending upon the composition of the particular sequence and composition of the particular database against which the sequence of interest is being searched. A percent (%) nucleic acid sequence identity value is determined by the number of matching identical nucleotides divided by the sequence length for which the percent identity is being reported.

Derivative subject nucleic acid sequences usually have at least 70% sequence identity, preferably at least 80% sequence identity, more preferably at least 85% sequence identity, still more preferably at least 90% sequence identity, and most preferably at least 95% sequence identity with SEQ ID NOs:1, 3, 5, 7, or 9 or domain-encoding regions thereof.

In one preferred embodiment, the derivative nucleic acids encode polypeptides comprising a subject amino acid sequences of SEQ ID NOs:2, 4, 6, 8, and 10 or fragments or derivatives thereof as described further below under the subheading “subject proteins”. A derivative subject nucleic acid sequence, or fragment thereof, may comprise 100% sequence identity with SEQ ID NOs:1, 3, 5, 7, or 9 but be a derivative thereof in the sense that it has one or more modifications at the base or sugar moiety, or phosphate backbone. Examples of modifications are well known in the art (Bailey, Ullmann's Encyclopedia of Industrial Chemistry (1998), 6th ed. Wiley and Sons). Such derivatives may be used to provide modified stability or any other desired property.

Another type of derivative of the subject nucleic acid sequences includes corresponding humanized sequences. A humanized nucleic acid sequence is one in which one or more codons has been substituted with a codon that is more commonly used in human genes. Preferably, a sufficient number of codons have been substituted such that a higher level expression is achieved in mammalian cells than what would otherwise be achieved without the substitutions. Tables are available that show, the codon frequency in humans for each amino acid (Wada et al., Nucleic Acids Research (1990) 18(Suppl.):2367-2411). Thus, a subject nucleic acid sequence in which the glutamic acid codon, GAA has been replaced with the codon GAG, which is more commonly used in human genes, is an example of a humanized subject nucleic acid sequence. A detailed discussion of the humanization of nucleic acid sequences is provided in U.S. Pat. No. 5,874,304 to Zolotukhin et al. Similarly, other nucleic acid derivatives can be generated with codon usage optimized for expression in other organisms, such as yeasts, bacteria, and plants, where it is desired to engineer the expression of subject proteins by using specific codons chosen according to the preferred codons used in highly expressed genes in each organism.

Nucleic acids encoding the amino acid sequence of any one of SEQ ID NOs:2, 4, 6, 8, or 10, or fragment or derivative thereof, may be obtained from an appropriate cDNA library prepared from any eukaryotic species that encodes subject proteins such as vertebrates, preferably mammalian (e.g. primate, porcine, bovine, feline, equine, and canine species, etc.) and invertebrates, such as arthropods, particularly insects species (preferably Drosophila), acarids, crustacea, molluscs, nematodes, and other worms. An expression library can be constructed using known methods. For example, mRNA can be isolated to make cDNA which is ligated into a suitable expression vector for expression in a host cell into which it is introduced. Various screening assays can then be used to select for the gene or gene product (e.g. oligonucleotides of at least about 20 to 80 bases designed to identify the gene of interest, or labeled antibodies that specifically bind to the gene product). The gene and/or gene product can then be recovered from the host cell using known techniques.

Polymerase chain reaction (PCR) can also be used to isolate nucleic acids of the subject where oligonucleotide primers representing fragmentary sequences of interest amplify RNA or DNA sequences from a source such as a genomic or cDNA library (as described by Sambrook et al., supra). Additionally, degenerate primers for amplifying homologs from any species of interest may be used. Once a PCR product of appropriate size and sequence is obtained, it may be cloned and sequenced by standard techniques, and utilized as a probe to isolate a complete cDNA or genomic clone.

Fragmentary sequences of subject nucleic acids and derivatives may be synthesized by known methods. For example, oligonucleotides may be synthesized using an automated DNA synthesizer available from commercial suppliers (e.g. Biosearch, Novato, Calif.; Perkin-Elmer Applied Biosystems, Foster City, Calif). Antisense RNA sequences can be produced intracellularly by transcription from an exogenous sequence, e.g. from vectors that contain antisense subject nucleic acid sequences. Newly generated sequences may be identified and isolated using standard methods.

An isolated subject nucleic acid sequence can be inserted into any appropriate cloning vector, for example bacteriophages such as lambda derivatives, or plasmids such as PBR322, pUC plasmid derivatives and the Bluescript vector (Stratagene, San Diego, Calif.). Recombinant molecules can be introduced into host cells via transformation, transfection, infection, electroporation, etc., or into a transgenic animal such as a fly. The transformed cells can be cultured to generate large quantities of the subject nucleic acid. Suitable methods for isolating and producing the subject nucleic acid sequences are well-known in the art (Sambrook et al., supra; DNA Cloning: A Practical Approach, Vol. 1, 2, 3, 4, (1995) Glover, ed., MRL Press, Ltd., Oxford, U.K.).

The nucleotide sequence encoding a subject protein or fragment or derivative thereof, can be inserted into any appropriate expression vector for the transcription and translation of the inserted protein-coding sequence. Alternatively, the necessary transcriptional and translational signals can be supplied by the native subject gene and/or its flanking regions. A variety of host-vector systems may be utilized to express the protein-coding sequence such as mammalian cell systems infected with virus (e.g. vaccinia virus, adenovirus, etc.); insect cell systems infected with virus (e.g. baculovirus); microorganisms such as yeast containing yeast vectors, or bacteria transformed with bacteriophage, DNA, plasmid DNA, or cosmid DNA. Expression of a subject protein may be controlled by a suitable promoter/enhancer element. In addition, a host cell strain may be selected which modulates the expression of the inserted sequences, or modifies and processes the gene product in the specific fashion desired.

To detect expression of a subject gene product, the expression vector can comprise a promoter operably linked to a subject gene nucleic acid, one or more origins of replication, and, one or more selectable markers (e.g. thymidine kinase activity, resistance to antibiotics, etc.). Alternatively, recombinant expression vectors can be identified by assaying for the expression of a subject gene product based on the physical or functional properties of a subject protein in in vitro assay systems (e.g. immunoassays).

A subject protein, fragment, or derivative may be optionally expressed as a fusion, or chimeric protein product (i.e. it is joined via a peptide bond to a heterologous protein sequence of a different protein). A chimeric product can be made by ligating the appropriate nucleic acid sequences encoding the desired amino acid sequences to each other in the proper coding frame using standard methods and expressing the chimeric product. A chimeric product may also be made by protein synthetic techniques, e.g. by use of a peptide synthesizer.

Once a recombinant that expresses a subject gene sequence is identified, the gene product can be isolated and purified using standard methods (e.g. ion exchange, affinity, and gel exclusion chromatography; centrifugation; differential solubility; electrophoresis). The amino acid sequence of the protein can be deduced from the nucleotide sequence of the chimeric gene contained in the recombinant and can thus be synthesized by standard chemical methods (Hunkapiller et al., Nature (1984) 310:105-111). Alternatively, native subject proteins can be purified from natural sources, by standard methods (e.g. immunoaffinity purification).

### Proteins of the Invention

Subject proteins of the invention comprise or consist of amino acid sequence of SEQ ID NOs:2, 4, 6, 8, and 10, or fragments or derivatives thereof. Compositions comprising these proteins may consist essentially of the subject protein, fragments, or derivatives, or may comprise additional components (e.g. pharmaceutically acceptable carriers or excipients, culture media, etc.).

Subject protein derivatives typically share a certain degree of sequence identity or sequence similarity with any of SEQ ID NOs:2, 4, 6, 8, or 10, or a fragment thereof. As used herein, “percent (%) amino acid sequence identity” with respect to a subject sequence, or a specified portion of a subject sequence, is defined as the percentage of amino acids in the candidate derivative amino acid sequence identical with the amino acid in the subject sequence (or specified portion thereof), after aligning the sequences and introducing gaps, if necessary to achieve the maximum percent sequence identity, as generated by BLAST (Altschul et al., supra) using the same parameters discussed above for derivative nucleic acid sequences. A % amino acid sequence identity value is determined by the number of matching identical amino acids divided by the sequence length for which the percent identity is being reported. “Percent (%) amino acid sequence similarity” is determined by doing the same calculation as for determining % amino acid sequence identity, but including conservative amino acid substitutions in addition to identical amino acids in the computation. A conservative amino acid substitution is one in which an amino acid is substituted for another amino acid having similar properties such that the folding or activity of the protein is not significantly affected. Aromatic amino acids that can be substituted for each other are phenylalanine, tryptophan, and tyrosine; interchangeable hydrophobic amino acids are leucine, isoleucine, methionine, and valine; interchangeable polar amino acids are glutamine and asparagine; interchangeable basic amino acids arginine, lysine and histidine; interchangeable acidic amino acids aspartic acid and glutamic acid; and interchangeable small amino acids alanine, serine, threonine, and glycine.

In one preferred embodiment, a subject protein derivative shares at least 80% sequence identity or similarity, preferably at least 85%, more preferably at least 90%, and most preferably at least 95% sequence identity or similarity with a contiguous stretch of at least 25 amino acids, preferably at least 50 amino acids, more preferably at least 100 amino acids, and in some cases, the entire length of any one of SEQ ID NO:2, 4, 6, 8, or 10.

The preferred dmAPS protein derivative may consist of or comprise a sequence that shares 100% similarity with any contiguous stretch of at least 200 amino acids, preferably at least 202 amino acids, more preferably at least 205 amino acids, and most preferably at least 210 amino acids of SEQ ID NO:2. Preferred derivatives of dmAPS consist of or comprise an amino acid sequence that has at least 80%, preferably at least 85%, more preferably at least 90%, and most preferably at least 95% sequence identity or sequence similarity with any of amino acid residues 285-307, or 442-519, which are the likely pleckstrin homology domain, and the SH2 domain, respectively. Preferred fragments of dmAPS proteins consist or comprise at least 202, preferably at least 204, more preferably at least 207, and most preferably at least 212 contiguous amino acids of SEQ ID NO:2.

The preferred dmCYP protein derivative may consist of or comprise a sequence that shares 100% similarity with any contiguous stretch of at least 16 amino acids, preferably at least 18 amino acids, more preferably at least 21 amino acids, and most preferably at least 26 amino acids of SEQ ID NO:4. Preferred fragments of dmCYP proteins consist or comprise at least 14, preferably at least 16, more preferably at least 19, and most preferably at least 24 contiguous amino acids of SEQ ID NO:4.

The preferred dmIGF protein derivative may consist of or comprise a sequence that shares 100% similarity with any contiguous stretch of at least 10 amino acids, preferably at least 12 amino acids, more preferably at least 15 amino acids, and most preferably at least 20 amino acids of SEQ ID NO:6. Preferred fragments of dmIGF proteins consist or comprise at least 5, preferably at least 7, more preferably at least 10, and most preferably at least 15 contiguous amino acids of SEQ ID NO:6.

The preferred dmSHIP2A protein derivative may consist of or comprise a sequence that shares 100% similarity with any contiguous stretch of at least 18 amino acids, preferably at least 20 amino acids, more preferably at least 23 amino acids, and most preferably at least 28 amino acids of SEQ ID NO:8. Preferred fragments of dmSHIP2A proteins consist or comprise at least 10, preferably at least 12, more preferably at least 15, and most preferably at least 20 contiguous amino acids of SEQ ID NO:8.

The preferred dmSHIP2B protein derivative may consist of or comprise a sequence that shares 100% similarity with any contiguous stretch of at least 38 amino acids, preferably at least 40 amino acids, more preferably at least 43 amino acids, and most preferably at least 48 amino acids of SEQ ID NO:10. Preferred fragments of dmSHIP2B proteins consist or comprise at least 20, preferably at least 22, more preferably at least 25, and most preferably at least 30 contiguous amino acids of SEQ ID NO:10.

The fragment or derivative of a subject protein is preferably “functionally active” meaning that the subject protein derivative or fragment exhibits one or more functional activities associated with a full-length, wild-type subject protein comprising the amino acid sequence of any of SEQ ID NOs:2, 4, 6, 8, or 10. As one example, a fragment or derivative may have antigenicity such that it can be used in immunoassays, for immunization, for inhibition of subject activity, etc, as discussed further below regarding generation of antibodies to subject proteins. Preferably, a functionally active dmAPS fragment or derivative is one that displays one or more biological activities associated with dmAPS proteins such as signaling activity. For purposes herein, functionally active fragments also include those fragments that exhibit one or more structural features of a dmAPS, such as pleckstrin homology, or SH2 domain. Preferably, a functionally active dmCYP fragment or derivative is one that displays one or more biological activities associated with dmCYP proteins such as enzymatic activity. For purposes herein, functionally active fragments also include those fragments that exhibit one or more structural features of a dmCYP, such as transmembrane domains. Preferably, a functionally active dmIGF fragment or derivative is one that displays one or more biological activities associated with dmIGF proteins, such as receptor binding. For purposes herein, functionally active fragments also include those fragments that exhibit one or more structural features of a dmIGF, such as the insulin family signature. Preferably, a functionally active dmSHIP2A or dmSHIP2B fragment or derivative is one that displays one or more biological activities associated with dmSHIP2A or dmSHIP2B proteins such as enzymatic activity. For purposes herein, functionally active fragments also include those fragments that exhibit one or more structural features or domains of a dmSHIP2A, such as an inositol polyphosphate phosphatase domain. The functional activity of subject proteins, derivatives and fragments can be assayed by various methods known to one skilled in the art (Current Protocols in Protein Science (1998) Coligan et al., eds., John Wiley & Sons, Inc., Somerset, N.J.). In a preferred method, which is described in detail below, a model organism, such as Drosophila, is used in genetic studies to assess the phenotypic effect of a fragment or derivative (i.e. a mutant subject protein).

Subject protein derivatives can be produced by various methods known in the art. The manipulations that result in their production can occur at the gene or protein level. For example, a cloned subject gene sequence can be cleaved at appropriate sites with restriction endonuclease(s) (Wells et al., Philos. Trans. R. Soc. London SerA (1986) 317:415), followed by further enzymatic modification if desired, isolated, and ligated in vitro, and expressed to produce the desired derivative. Alternatively, a subject gene can be mutated in vitro or in vivo, to create and/or destroy translation, initiation, and/or termination sequences, or to create variations in coding regions and/or to form new restriction endonuclease sites or destroy preexisting ones, to facilitate further in vitro modification. A variety of mutagenesis techniques are known in the art such as chemical mutagenesis, in vitro site-directed mutagenesis (Carter et al., Nucl. Acids Res. (1986) 13:4331), use of TAB® linkers (available from Pharmacia and Upjohn, Kalamazoo, Mich.), etc.

At the protein level, manipulations include post translational modification, e.g. glycosylation, acetylation, phosphorylation, amidation, derivatization by known protecting/blocking groups, proteolytic cleavage, linkage to an antibody molecule or other cellular ligand, etc. Any of numerous chemical modifications may be carried out by known technique (e.g. specific chemical cleavage by cyanogen bromide, trypsin, chymotrypsin, papain, V8 protease, NaBH₄, acetylation, formylation, oxidation, reduction, metabolic synthesis in the presence of tunicamycin, etc.). Derivative proteins can also be chemically synthesized by use of a peptide synthesizer, for example to introduce nonclassical amino acids or chemical amino acid analogs as substitutions or additions into the subject protein sequence.

Chimeric or fusion proteins can be made comprising a subject protein or fragment thereof (preferably comprising one or more structural or functional domains of the subject protein) joined at its amino- or carboxy-terminus via a peptide bond to an amino acid sequence of a different protein. Chimeric proteins can be produced by any known method, including: recombinant expression of a nucleic acid encoding the protein (comprising a subject-coding sequence joined in-frame to a coding sequence for a different protein); ligating the appropriate nucleic acid sequences encoding the desired amino acid sequences to each other in the proper coding frame, and expressing the chimeric product; and protein synthetic techniques, e.g. by use of a peptide synthesizer.

### Subject Gene Regulatory Elements

Subject genes' regulatory DNA elements, such as enhancers or promoters, can be used to identify tissues, cells, genes and factors that specifically control subject protein production. For example, such regulatory elements reside within nucleotides 1 to 446 of SEQ ID NO:1 (dmAPS), within nucleotides 1 to 77 of SEQ ID NO:5 (dmIGF), within nucleotides 1 to 234 of SEQ ID NO:7 (dmSHIP2A), and within nucleotides 1 to 213 of SEQ ID NO:9 (dmSHIP2B). Preferably at least 20, more preferably at least 25, and most preferably at least 50 contiguous nucleotides within any of these fragments are used. Analyzing components that are specific to subject protein function can lead to an understanding of how to manipulate these regulatory processes, especially for therapeutic applications, as well as an understanding of how to diagnose dysfunction in these processes.

Gene fusions with the subject regulatory elements can be made. For compact genes that have relatively few and small intervening sequences, such as those described herein for Drosophila, it is typically the case that the regulatory elements that control spatial and temporal expression patterns are found in the DNA immediately upstream of the coding region, extending to the nearest neighboring gene. Regulatory regions can be used to construct gene fusions where the regulatory DNAs are operably fused to a coding region for a reporter protein whose expression is easily detected, and these constructs are introduced as transgenes into the animal of choice. An entire regulatory DNA region can be used, or the regulatory region can be divided into smaller segments to identify sub-elements that might be specific for controlling expression a given cell type or stage of development. Reporter proteins that can be used for construction of these gene fusions include E. coli beta-galactosidase and green fluorescent protein (GFP). These can be detected readily in situ, and thus are useful for histological studies and can be used to sort cells that express subject proteins (O'Kane and Gehring PNAS (1987) 84(24):9123-9127; Chalfie et al., Science (1994) 263:802-805; and Cumberledge and Krasnow (1994) Methods in Cell Biology 44:143-159). Recombinase proteins, such as FLP or cre, can be used in controlling gene expression through site-specific recombination (Golic and Lindquist (1989) Cell 59(3):499-509; White et al., Science (1996) 271:805-807). Toxic proteins such as the reaper and hid cell death proteins, are useful to specifically ablate cells that normally express subject proteins in order to assess the physiological function of the cells (Kingston, In Current Protocols in Molecular Biology (1998) Ausubel et al., John Wiley & Sons, Inc. sections 12.0.3-12.10) or any other protein where it is desired to examine the function this particular protein specifically in cells that synthesize subject proteins.

Alternatively, a binary reporter system can be used, similar to that described further below, where the subject regulatory element is operably fused to the coding region of an exogenous transcriptional activator protein, such as the GAL4 or tTA activators described below, to create a subject regulatory element “driver gene”. For the other half of the binary system the exogenous activator controls a separate “target gene” containing a coding region of a reporter protein operably fused to a cognate regulatory element for the exogenous activator protein, such as UAS_{G} or a tTA-response element, respectively. An advantage of a binary system is that a single driver gene construct can be used to activate transcription from preconstructed target genes encoding different reporter proteins, each with its own uses as delineated above.

Subject regulatory element-reporter gene fusions are also useful for tests of genetic interactions, where the objective is to identify those genes that have a specific role in controlling the expression of subject genes, or promoting the growth and differentiation of the tissues that expresses the subject protein. Subject gene regulatory DNA elements are also useful in protein-DNA binding assays to identify gene regulatory proteins that control the expression of subject genes. The gene regulatory proteins can be detected using a variety of methods that probe specific protein-DNA interactions well known to those skilled in the art (Kingston, supra) including in vivo footprinting assays based on protection of DNA sequences from chemical and enzymatic modification within living or permeabilized cells; and in vitro footprinting assays based on protection of DNA sequences from chemical or enzymatic modification using protein extracts, nitrocellulose filter-binding assays and gel electrophoresis mobility shift assays using radioactively labeled regulatory DNA elements mixed with protein extracts. Candidate subject gene regulatory proteins can be purified using a combination of conventional and DNA-affinity purification techniques. Molecular cloning strategies can also be used to identify proteins that specifically bind subject gene regulatory DNA elements. For example, a Drosophila cDNA library in an expression vector, can be screened for cDNAs that encode subject gene regulatory element DNA-binding activity. Similarly, the yeast “one-hybrid” system can be used (Li and Herskowitz, Science (1993) 262:1870-1874; Luo et al., Biotechniques (1996) 20(4):564-568; Vidal et al., PNAS (1996) 93(19):10315-10320).

### Antibodies and Immunoassays

Subject proteins encoded by SEQ ID NOs:2, 4, 6, 8, and 10 and derivatives and fragments thereof, such as those discussed above, may be used as an immunogen to generate monoclonal or polyclonal antibodies and antibody fragments or derivatives (e.g. chimeric, single chain, Fab fragments). For example, fragments of a subject protein, preferably those identified as hydrophilic, are used as immunogens for antibody production using art-known methods such as by hybridomas; production of monoclonal antibodies in germ-free animals (PCT/US90/02545); the use of human hybridomas (Cole et al., PNAS (1983) 80:2026-2030; Cole et al., in Monoclonal Antibodies and Cancer Therapy (1985) Alan R. Liss, pp. 77-96), and production of humanized antibodies (Jones et al., Nature (1986) 321:522-525; U.S. Pat. No. 5,530,101). In a particular embodiment, subject polypeptide fragments provide specific antigens and/or immunogens, especially when coupled to carrier proteins. For example, peptides are covalently coupled to keyhole limpet antigen (KLH) and the conjugate is emulsified in Freund's complete adjuvant. Laboratory rabbits are immunized according to conventional protocol and bled. The presence of specific antibodies is assayed by solid phase immunosorbent assays using immobilized corresponding polypeptide. Specific activity or function of the antibodies produced may be determined by convenient in vitro, cell-based, or in vivo assays: e.g. in vitro binding assays, etc. Binding affinity may be assayed by determination of equilibrium constants of antigen-antibody association (usually at least about 10⁷ M⁻¹, preferably at least about 10⁸ M⁻¹, more preferably at least about 10⁹ M⁻¹).

Immunoassays can be used to identify proteins that interact with or bind to subject protein. Various assays are available for testing the ability of a protein to bind to or compete with binding to a wild-type subject protein or for binding to an anti-subject protein antibody. Suitable assays include radioimmunoassays, ELISA (enzyme linked immunosorbent assay), immunoradiometric assays, gel diffusion precipitin reactions, immunodiffusion assays, in situ immunoassays (e.g., using colloidal gold, enzyme or radioisotope labels), western blots, precipitation reactions, agglutination assays (e.g., gel agglutination assays, hemagglutination assays), complement fixation assays, immunofluorescence assays, protein A assays, immunoelectrophoresis assays, etc.

### Identification of Molecules that Interact With Subject Proteins

A variety of methods can be used to identify or screen for molecules, such as proteins or other molecules, that interact with subject protein, or derivatives or fragments thereof. The assays may employ purified subject protein, or cell lines or model organisms such as Drosophila and C. elegans, which have been genetically engineered to express subject protein. Suitable screening methodologies are well known in the art to test for proteins and other molecules that interact with subject gene and protein (see e.g., PCT International Publication No. WO 96/34099). The newly identified interacting molecules may provide new targets for pharmaceutical agents. Any of a variety of exogenous molecules, both naturally occurring and/or synthetic (e.g., libraries of small molecules or peptides, or phage display libraries), may be screened for binding capacity. In a typical binding experiment, the subject protein or fragment is mixed with candidate molecules under conditions conducive to binding, sufficient time is allowed for any binding to occur, and assays are performed to test for bound complexes. Assays to find interacting proteins can be performed by any method known in the art, for example, immunoprecipitation with an antibody that binds to the protein in a complex followed by analysis by size fractionation of the immunoprecipitated proteins (e.g. by denaturing or nondenaturing polyacrylamide gel electrophoresis), Western analysis, non-denaturing gel electrophoresis, etc.

### Two-hybrid Assay Systems

A preferred method for identifying interacting proteins is a two-hybrid assay system or variation thereof (Fields and Song, Nature (1989) 340:245-246; U.S. Pat. No. 5,283,173; for review see Brent and Finley, Annu. Rev. Genet. (1997) 31:663-704). The most commonly used two-hybrid screen system is performed using yeast. All systems share three elements: 1) a gene that directs the synthesis of a “bait” protein fused to a DNA binding domain; 2) one or more “reporter” genes having an upstream binding site for the bait, and 3) a gene that directs the synthesis of a “prey” protein fused to an activation domain that activates transcription of the reporter gene. For the screening of proteins that interact with subject protein, the “bait” is preferably a subject protein, expressed as a fusion protein to a DNA binding domain; and the “prey” protein is a protein to be tested for ability to interact with the bait, and is expressed as a fusion protein to a transcription activation domain. The prey proteins can be obtained from recombinant biological libraries expressing random peptides.

The bait fusion protein can be constructed using any suitable DNA binding domain, such as the E. coli LexA repressor protein, or the yeast GAL4 protein (Bartel et al., BioTechniques (1993) 14:920-924, Chasman et al., Mol. Cell. Biol. (1989) 9:4746-4749; Ma et al., Cell (1987) 48:847-853; Ptashne et al., Nature (1990) 346:329-331).

The prey fusion protein can be constructed using any suitable activation domain such as GAL4, VP-16, etc. The preys may contain useful moieties such as nuclear localization signals (Ylikomi et al., EMBO J. (1992) 11:3681-3694; Dingwall and Laskey, Trends Biochem. Sci. Trends Biochem. Sci. (1991) 16:479-481) or epitope tags (Allen et al., Trends Biochem. Sci. Trends Biochem. Sci. (1995) 20:511-516) to facilitate isolation of the encoded proteins.

Any reporter gene can be used that has a detectable phenotype such as reporter genes that allow cells expressing them to be selected by growth on appropriate medium (e.g. HIS3, LEU2 described by Chien et al., PNAS (1991) 88:9572-9582; and Gyuris et al., Cell (1993) 75:791-803). Other reporter genes, such as LacZ and GFP, allow cells expressing them to be visually screened (Chien et al., supra).

Although the preferred host for two-hybrid screening is the yeast, the host cell in which the interaction assay and transcription of the reporter gene occurs can be any cell, such as mammalian (e.g. monkey, mouse, rat, human, bovine), chicken, bacterial, or insect cells. Various vectors and host strains for expression of the two fusion protein populations in yeast can be used (U.S. Pat. No. 5,468,614; Bartel et al., Cellular Interactions in Development (1993) Hartley, ed., Practical Approach Series xviii, IRL Press at Oxford University Press, New York, N.Y., pp. 153-179; and Fields and Sternglanz, Trends In Genetics (1994) 10:286-292). As an example of a mammalian system, interaction of activation tagged VP16 derivatives with a GAL4-derived bait drives expression of reporters that direct the synthesis of hygromycin B phosphotransferase, chloramphenicol acetyltransferase, or CD4 cell surface antigen (Fearon et al., PNAS (1992) 89:7958-7962). As another example, interaction of VP16-tagged derivatives with GAL4-derived baits drives the synthesis of SV40 T antigen, which in turn promotes the replication of the prey plasmid, which carries an SV40 origin (Vasavada et al., PNAS (1991) 88:10686-10690).

Typically, the bait subject gene and the prey library of chimeric genes are combined by mating the two yeast strains on solid or liquid media for a period of approximately 6-8 hours. The resulting diploids contain both kinds of chimeric genes, i.e., the DNA-binding domain fusion and the activation domain fusion.

Transcription of the reporter gene can be detected by a linked replication assay in the case of SV40 T antigen (described by Vasavada et al., supra) or using immunoassay methods, preferably as described in Alam and Cook (Anal. Biochem. (1990)188:245-254). The activation of other reporter genes like URA3, HIS3, LYS2, or LEU2 enables the cells to grow in the absence of uracil, histidine, lysine, or leucine, respectively, and hence serves as a selectable marker. Other types of reporters are monitored by measuring a detectable signal. For example, GFP and lacZ have gene products that are fluorescent and chromogenic, respectively.

After interacting proteins have been identified, the DNA sequences encoding the proteins can be isolated. In one method, the activation domain sequences or DNA-binding domain sequences (depending on the prey hybrid used) are amplified, for example, by PCR using pairs of oligonucleotide primers specific for the coding region of the DNA binding domain or activation domain. Other known amplification methods can be used, such as ligase chain reaction, use of Q replicase, or various other methods described (see Kricka et al., Molecular Probing, Blotting, and Sequencing (1995) Academic Press, New York, Chapter 1 and Table IX).

If a shuttle (yeast to E. coli) vector is used to express the fusion proteins, the DNA sequences encoding the proteins can be isolated by transformation of E. coli using the yeast DNA and recovering the plasmids from E. coli. Alternatively, the yeast vector can be isolated, and the insert encoding the fusion protein subcloned into a bacterial expression vector, for growth of the plasmid in E. coli.

A limitation of the two-hybrid system occurs when transmembrane portions of proteins in the bait or the prey fusions are used. This occurs because most two-hybrid systems are designed to function by formation of a functional transcription activator complex within the nucleus, and use of transmembrane portions of the protein can interfere with proper association, folding, and nuclear transport of bait or prey segments (Ausubel et al., supra; Allen et al., supra). Since the dmCYP, dmSHIP2A, and dmSHIP2B proteins are transmembrane proteins, it is preferred that intracellular or extracellular domains be used for bait in a two-hybrid scheme.

### Identification of Potential Drug Targets

Once new subject genes or subject interacting genes are identified, they can be assessed as potential drug targets. Putative drugs and molecules can be applied onto whole insects, nematodes, and other small invertebrate metazoans, and the ability of the compounds to modulate (e.g. block or enhance) subject activity can be observed. Alternatively, the effect of various compounds on subjects can be assayed using cells that have been engineered to express one or more subjects and associated proteins.

### Assays of Compounds on Worms

In a typical worm assay, the compounds to be tested are dissolved in DMSO or other organic solvent, mixed with a bacterial suspension at various test concentrations, preferably OP50 strain of bacteria (Brenner, Genetics (1974) 110:421-440), and supplied as food to the worms. The population of worms to be treated can be synchronized larvae (Sulston and Hodgkin, in The nematode C. elegans (1988), supra) or adults or a mixed-stage population of animals.

Adult and larval worms are treated with different concentrations of compounds, typically ranging from 1 mg/ml to 0.001 mg/ml. Behavioral aberrations, such as a decrease in motility and growth, and morphological aberrations, sterility, and death are examined in both acutely and chronically treated adult and larval worms. For the acute assay, larval and adult worms are examined immediately after application of the compound and re-examined periodically (every 30 minutes) for 5-6 hours. Chronic or long-term assays are performed on worms and the behavior of the treated worms is examined every 8-12 hours for 4-5 days. In some circumstances, it is necessary to reapply the compound to the treated worms every 24 hours for maximal effect.

### Assays of Compounds on Insects

Potential insecticidal compounds can be administered to insects in a variety of ways, including orally (including addition to synthetic diet, application to plants or prey to be consumed by the test organism), topically (including spraying, direct application of compound to animal, allowing animal to contact a treated surface), or by injection. Insecticides are typically very hydrophobic molecules and must commonly be dissolved in organic solvents, which are allowed to evaporate in the case of methanol or acetone, or at low concentrations can be included to facilitate uptake (ethanol, dimethyl sulfoxide).

The first step in an insect assay is usually the determination of the minimal lethal dose (MLD) on the insects after a chronic exposure to the compounds. The compounds are usually diluted in DMSO, and applied to the food surface bearing 0-48 hour old embryos and larvae. In addition to MLD, this step allows the determination of the fraction of eggs that hatch, behavior of the larvae, such as how they move/feed compared to untreated larvae, the fraction that survive to pupate, and the fraction that eclose (emergence of the adult insect from puparium). Based on these results more detailed assays with shorter exposure times may be designed, and larvae might be dissected to look for obvious morphological defects. Once the MLD is determined, more specific acute and chronic assays can be designed.

In a typical acute assay, compounds are applied to the food surface for embryos, larvae, or adults, and the animals are observed after 2 hours and after an overnight incubation. For application on embryos, defects in development and the percent that survive to adulthood are determined. For larvae, defects in behavior, locomotion, and molting may be observed. For application on adults, behavior and neurological defects are observed, and effects on fertility are noted.

For a chronic exposure assay, adults are placed on vials containing the compounds for 48 hours, then transferred to a clean container and observed for fertility, neurological defects, and death.

### Assay of Compounds using Cell Cultures

Compounds that modulate (e.g. block or enhance) subject activity may also be assayed using cell culture. For example, various compounds added to cells expressing dmAPS may be screened for their ability to modulate the activity of dmAPS genes based upon measurements of in vitro interactions. Alternatively, various compounds added to cells expressing dmCYP, dmSHIP2A, or dmSHIP2B may be screened for their ability to modulate the activity of dmCYP, dmSHIP2A, or dmSHIP2B genes based upon measurements of these proteins' enzymatic activity. Alternatively still, various compounds added to cells expressing dmIGF may be screened for their ability to modulate the activity of dmIGF genes based upon measurements of receptor binding or mitogenic activity. Assays for changes in subject gene function can be performed on cultured cells expressing endogenous normal or mutant subjects. Such studies also can be performed on cells transfected with vectors capable of expressing the subject genes, or functional domains of one of the subjects, in normal or mutant form. In addition, to enhance the signal measured in such assays, cells may be cotransfected with genes encoding subject proteins.

As an example, full-length and subdomains of APS are subcloned into expression plasmid pGEX5X (Amersham Pharmacia Biotech, Piscataway, N.J.), and interaction studies are performed as described (Moodie SA et al., J Biol Chem,(1999) 274 11186-11193, and also described below in Example 4), in presence or absence of compounds.

As another example, native or modified dmCYP may be expressed and then purified from cells. Measuring dmCYP activity can then be accomplished by measuring the consumption of oxygen, or by measuring the consumption of NADPH or NADH by the redox partner. Measuring dmCYP inhibition is frequently done by designing substrate probes that yield a fluorescent signal upon activation (e.g. O-demethylation) by the dmCYP. Then test compounds can be asssayed for their ability to inhibit the production of the fluorescent signal asunder controlled conditions.

As another example, the dmIGF purified protein may be added to cells and assayed for mitogenic effects, as described in the Examples, below.

As another example, dmSHIP2A or dmSHIP2B may be transfected into cells, and cell extracts may be used to assess the phospahatase activity on relevant substrates.

Compounds that selectively modulate the subject gene activity are identified as potential drug candidates having subject specificity. Identification of small molecules and compounds as potential pharmaceutical compounds from large chemical libraries requires high-throughput screening (HTS) methods (Bolger, Drug Discovery Today (1999) 4:251-253). Several of the assays mentioned herein can lend themselves to such screening methods. For example, cells or cell lines expressing wild type or mutant subject protein or its fragments, and a reporter gene can be subjected to compounds of interest, and depending on the reporter genes, interactions can be measured using a variety of methods such as color detection, fluorescence detection (e.g. GFP), autoradiography, scintillation analysis, etc.

### Generation and Genetic Analysis of Animals and Cell Lines with Altered Expression of Subject Gene

Both genetically modified animal models (i.e. in vivo models), such as C. elegans and Drosophila, and in vitro models such as genetically engineered cell lines expressing or mis-expressing subject pathway genes, are useful for the functional analysis of these proteins. Model systems that display detectable phenotypes, can be used for the identification and characterization of subject pathway genes or other genes of interest and/or phenotypes associated with the mutation or mis-expression of subject pathway protein. The term “mis-expression” as used herein encompasses mis-expression due to gene mutations. Thus, a mis-expressed subject pathway protein may be one having an amino acid sequence that differs from wild-type (i.e. it is a derivative of the normal protein). A mis-expressed subject pathway protein may also be one in which one or more amino acids have been deleted, and thus is a “fragment” of the normal protein. As used herein, “mis-expression” also includes ectopic expression (e.g. by altering the normal spatial or temporal expression), over-expression (e.g. by multiple gene copies), underexpression, non-expression (e.g. by gene knockout or blocking expression that would otherwise normally occur), and further, expression in ectopic tissues. As used in the following discussion concerning in vivo and in vitro models, the term “gene of interest” refers to a subject pathway gene, or any other gene involved in regulation or modulation, or downstream effector of the subject pathway.

The in vivo and in vitro models may be genetically engineered or modified so that they 1) have deletions and/or insertions of one or more subject pathway genes, 2) harbor interfering RNA sequences derived from subject pathway genes, 3) have had one or more endogenous subject pathway genes mutated (e.g. contain deletions, insertions, rearrangements, or point mutations in subject gene or other genes in the pathway), and/or 4) contain transgenes for mis-expression of wild-type or mutant forms of such genes. Such genetically modified in vivo and in vitro models are useful for identification of genes and proteins that are involved in the synthesis, activation, control, etc. of subject pathway gene and/or gene products, and also downstream effectors of subject function, genes regulated by subject, etc. The model systems can also be used for testing potential pharmaceutical compounds that interact with the subject pathway, for example by administering the compound to the model system using any suitable method (e.g. direct contact, ingestion, injection, etc.) and observing any changes in phenotype, for example defective movement, lethality, etc. Various genetic engineering and expression modification methods which can be used are well-known in the art, including chemical mutagenesis, transposon mutagenesis, antisense RNAi, dsRNAi, and transgene-mediated mis-expression.

### Generating Loss-of-function Mutations by Mutagenesis

Loss-of-function mutations in an invertebrate metazoan subject gene can be generated by any of several mutagenesis methods known in the art (Ashbumer, In Drosophila melanogaster: A Laboratory Manual (1989), Cold Spring Harbor, N.Y., Cold Spring Harbor Laboratory Press: pp. 299-418; Fly pushing: The Theory and Practice of Drosophila melanogaster Genetics (1997) Cold Spring Harbor Press, Plainview, N.Y.; The nematode C. elegans (1988) Wood, Ed., Cold Spring Harbor Laboratory Press, Cold Spring harbor, N.Y. Techniques for producing mutations in a gene or genome include use of radiation ( e.g., X-ray, UV, or gamma ray); chemicals (e.g., EMS, MMS, ENU, formaldehyde, etc.); and insertional mutagenesis by mobile elements including dysgenesis induced by transposon insertions, or transposon-mediated deletions, for example, male recombination, as described below. Other methods of altering expression of genes include use of transposons (e.g., P element, EP-type “overexpression trap” element, mariner element, piggyBac transposon, hermes, minos, sleeping beauty, etc.) to misexpress genes; antisense; double-stranded RNA interference; peptide and RNA aptamers; directed deletions; homologous recombination; dominant negative alleles; and intrabodies.

Transposon insertions lying adjacent to a gene of interest can be used to generate deletions of flanking genomic DNA, which if induced in the germline, are stably propagated in subsequent generations. The utility of this technique in generating deletions has been demonstrated and is well-known in the art. One version of the technique using collections of P element transposon induced recessive lethal mutations (P lethals) is particularly suitable for rapid identification of novel, essential genes in Drosophila (Cooley et al., Science (1988) 239:1121-1128; Spralding et al., PNAS (1995) 92:0824-10830). Since the sequence of the P elements are known, the genomic sequence flanking each transposon insert is determined either by plasmid rescue (Hamilton et al., PNAS (1991) 88:2731-2735) or by inverse polymerase chain reaction (Rehm, http://www.fruitfly.org/methods/).

A more recent version of the transposon insertion technique in male Drosophila using P elements is known as P-mediated male recombination (Preston and Engels, Genetics (1996) 144:1611-1638).

### Generating Loss-of-function Phenotypes Using RNA-based Methods

Subject genes may be identified and/or characterized by generating loss-of-function phenotypes in animals of interest through RNA-based methods, such as antisense RNA (Schubiger and Edgar, Methods in Cell Biology (1994) 44:697-713). One form of the antisense RNA method involves the injection of embryos with an antisense RNA that is partially homologous to the gene of interest (in this case the subject gene). Another form of the antisense RNA method involves expression of an antisense RNA partially homologous to the gene of interest by operably joining a portion of the gene of interest in the antisense orientation to a powerful promoter that can drive the expression of large quantities of antisense RNA, either generally throughout the animal or in specific tissues. Antisense RNA-generated loss-of-function phenotypes have been reported previously for several Drosophila genes including cactus, pecanex, and Krüppel (LaBonne et al., Dev. Biol. (1989) 136(1):1-16; Schuh and Jackie, Genome (1989) 31(1):422-425; Geisler et al., Cell (1992) 71(4):613-621).

Loss-of-function phenotypes can also be generated by cosuppression methods (Bingham Cell (1997) 90(3):385-387; Smyth, Curr. Biol. (1997) 7(12):793-795; Que and Jorgens, Dev. Genet. (1998) 22(1): 100-109). Cosuppression is a phenomenon of reduced gene expression produced by expression or injection of a sense strand RNA corresponding to a partial segment of the gene of interest. Cosuppression effects have been employed extensively in plants and C. elegans to generate loss-of-function phenotypes, and there is a single report of cosuppression in Drosophila, where reduced expression of the Adh gene was induced from a white-Adh transgene using cosuppression methods (Pal-Bhadra et al., Cell (1997) 90(3):479-490).

Another method for generating loss-of-function phenotypes is by double-stranded RNA interference (dsRNAi). This method is based on the interfering properties of double-stranded RNA derived from the coding regions of gene, and has proven to be of great utility in genetic studies of C. elegans (Fire et al., Nature (1998) 391:806-811), and can also be used to generate loss-of-function phenotypes in Drosophila (Kennerdell and Carthew, Cell (1998) 95:1017-1026; Misquitta and Patterson PNAS (1999) 96:1451-1456). In one example of this method, complementary sense and antisense RNAs derived from a substantial portion of a gene of interest, such as subject gene, are synthesized in vitro. The resulting sense and antisense RNAs are annealed in an injection buffer, and the double-stranded RNA injected or otherwise introduced into animals (such as in their food or by soaking in the buffer containing the RNA). Progeny of the injected animals are then inspected for phenotypes of interest (PCT publication no. WO99/32619). In another embodiment of the method, the dsRNA can be delivered to the animal by bathing the animal in a solution containing a sufficient concentration of the dsRNA. In another embodiment of the method, dsRNA derived from subject genes can be generated in vivo by simultaneous expression of both sense and antisense RNA from appropriately positioned promoters operably fused to subject sequences in both sense and antisense orientations. In yet another embodiment of the method the dsRNA can be delivered to the animal by engineering expression of dsRNA within cells of a second organism that serves as food for the animal, for example engineering expression of dsRNA in E. coli bacteria which are fed to C. elegans, or engineering expression of dsRNA in baker's yeast which are fed to Drosophila, or engineering expression of dsRNA in transgenic plants which are fed to plant eating insects such as Leptinotarsa or Heliothis.

Recently, RNAi has been successfully used in cultured Drosophila cells to inhibit expression of targeted proteins (Dixon lab, University of Michigan, http://dixonlab.biochem.med.umich.edu/protocols/RNAiExperiments.html; Caplen et al., Gene. (2000) 252(1-2):95-105). Thus, cell lines in culture can be manipulated using RNAi both to perturb and study the function of subject pathway components and to validate the efficacy of therapeutic strategies that involve the manipulation of this pathway.

### Generating Loss-of-function Phenotypes Using Peptide and RNA Aptamers

Another method for generating loss-of-function phenotypes is by the use of peptide aptamers, which are peptides or small polypeptides that act as dominant inhibitors of protein function. Peptide aptamers specifically bind to target proteins, blocking their function ability (Kolonin and Finley, PNAS (1998) 95:14266-14271). Due to the highly selective nature of peptide aptamers, they may be used not only to target a specific protein, but also to target specific functions of a given protein (e.g. signaling function of dmAPS, mitotic function of dmIGF, or enzymatic function of dmCYP, dmSHIP2A, or dmSHIP2B). Further, peptide aptamers may be expressed in a controlled fashion by use of promoters which regulate expression in a temporal, spatial or inducible manner. Peptide aptamers act dominantly; therefore, they can be used to analyze proteins for which loss-of-function mutants are not available.

Peptide aptamers that bind with high affinity and specificity to a target protein may be isolated by a variety of techniques known in the art. In one method, they are isolated from random peptide libraries by yeast two-hybrid screens (Xu et al., PNAS (1997) 94:12473-12478). They can also be isolated from phage libraries (Hoogenboom et al., Immunotechnology (1998) 4:1-20) or chemically generated peptides/libraries.

RNA aptamers are specific RNA ligands for proteins, that can specifically inhibit protein function of the gene (Good et al., Gene Therapy (1997) 4:45-54; Ellington. et al., Biotechnol. Annu. Rev. (1995) 1:185-214). In vitro selection methods can be used to identify RNA aptamers having a selected specificity (Bell et al., J. Biol. Chem. (1998) 273:14309-14314). It has been demonstrated that RNA aptamers can inhibit protein function in Drosophila (Shi et al., Proc. Natl. Acad. Sci USA (19999) 96:10033-10038). Accordingly, RNA aptamers can be used to decrease the expression of subject protein or derivative thereof, or a protein that interacts with the subject protein.

Transgenic animals can be generated to test peptide or RNA aptamers in vivo (Kolonin, M G, and Finley, R L, Genetics, 1998 95:4266-4271). For example, transgenic Drosophila lines expressing the desired aptamers may be generated by P element mediated transformation (discussed below). The phenotypes of the progeny expressing the aptamers can then be characterized.

### Generating Loss of Function Phenotypes Using Intrabodies

Intracellularly expressed antibodies, or intrabodies, are single-chain antibody molecules designed to specifically bind and inactivate target molecules inside cells. Intrabodies have been used in cell assays and in whole organisms such as Drosophila (Chen et al., Hum. Gen. Ther. (1994) 5:595-601; Hassanzadeh et al., Febs Lett. (1998) 16(1, 2):75-80 and 81-86). expression vectors can be constructed with intrabodies that react specifically with subject protein. These vectors can be introduced into model organisms and studied in the same manner as described above for aptamers.

### Transgenesis

Typically, transgenic animals are created that contain gene fusions of the coding regions of the subject gene (from either genomic DNA or cDNA) or genes engineered to encode antisense RNAs, cosuppression RNAs, interfering dsRNA, RNA aptamers, peptide aptamers, or intrabodies operably joined to a specific promoter and transcriptional enhancer whose regulation has been well characterized, preferably heterologous promoters/enhancers (i.e. promoters/enhancers that are non-native to the subject pathway genes being expressed).

Methods are well known for incorporating exogenous nucleic acid sequences into the genome of animals or cultured cells to create transgenic animals or recombinant cell lines. For invertebrate animal models, the most common methods involve the use of transposable elements. There are several suitable transposable elements that can be used to incorporate nucleic acid sequences into the genome of model organisms. Transposable elements are particularly useful for inserting sequences into a gene of interest so that the encoded protein is not properly expressed, creating a “knock-out” animal having a loss-of-function phenotype. Techniques are well-established for the use of P element in Drosophila (Rubin and Spradling, Science (1982) 218:348-53; U.S. Pat. No. 4,670,388) and Tc1 in C. elegans (Zwaal et al., Proc. Natl. Acad. Sci. U.S.A. (1993) 90:7431-7435; and Caenorhabditis elegans: Modern Biological Analysis of an Organism (1995) Epstein and Shakes, Eds.). Other Tc1-like transposable elements can be used such as minos, mariner and sleeping beauty. Additionally, transposable elements that function in a variety of species, have been identified, such as PiggyBac (Thibault et al., Insect Mol Biol (1999) 8(1):119-23), hobo, and hermes.

P elements, or marked P elements, are preferred for the isolation of loss-of-function mutations in Drosophila subject genes because of the precise molecular mapping of these genes, depending on the availability and proximity of preexisting P element insertions for use as a localized transposon source (Hamilton and Zinn, Methods in Cell Biology (1994) 44:81-94; and Wolfner and Goldberg, Methods in Cell Biology (1994) 44:33-80). Typically, modified P elements are used which contain one or more elements that allow detection of animals containing the P element. Most often, marker genes are used that affect the eye color of Drosophila, such as derivatives of the Drosophila white or rosy genes (Rubin and Spradling, Science (1982) 218(4570):348-353; and Klemenz et al., Nucleic Acids Res. (1987) 15(10):3947-3959). However, in principle, any gene can be used as a marker that causes a reliable and easily scored phenotypic change in transgenic animals. Various other markers include bacterial plasmid sequences having selectable markers such as ampicillin resistance (Steller and Pirrotta, EMBO. J. (1985) 4:167-171); and lacZ sequences fused to a weak general promoter to detect the presence of enhancers with a developmental expression pattern of interest (Bellen et al., Genes Dev. (1989) 3(9):1288-1300). Other examples of marked P elements useful for mutagenesis have been reported (Nucleic Acids Research (1998) 26:85-88; and http://flybase.bio.indiana.edu).

A preferred method of transposon mutagenesis in Drosophila employs the “local hopping” method described by Tower et al. (Genetics (1993) 133:347-359). Each new P insertion line can be tested molecularly for transposition of the P element into the gene of interest by assays based on PCR. For each reaction, one PCR primer is used that is homologous to sequences contained within the P element and a second primer is homologous to the coding region or flanking regions of the gene of interest. Products of the PCR reactions are detected by agarose gel electrophoresis. The sizes of the resulting DNA fragments reveal the site of P element insertion relative to the gene of interest. Alternatively, Southern blotting and restriction mapping using DNA probes derived from genomic DNA or cDNAs of the gene of interest can be used to detect transposition events that rearrange the genomic DNA of the gene. P transposition events that map to the gene of interest can be assessed for phenotypic effects in heterozygous or homozygous mutant Drosophila.

In another embodiment, Drosophila lines carrying P insertions in the gene of interest, can be used to generate localized deletions using known methods (Kaiser, Bioassays (1990) 12(6):297-301; Harnessing the power of Drosophila genetics, In Drosophila melanogaster: Practical Uses in Cell and Molecular Biology, Goldstein and Fyrberg, Eds., Academic Press, Inc. San Diego, Calif.). This is particularly useful if no P element transpositions are found that disrupt the gene of interest. Briefly, flies containing P elements inserted near the gene of interest are exposed to a further round of transposase to induce excision of the element. Progeny in which the transposon has excised are typically identified by loss of the eye color marker associated with the transposable element. The resulting progeny will include flies with either precise or imprecise excision of the P element, where the imprecise excision events often result in deletion of genomic DNA neighboring the site of P insertion. Such progeny are screened by molecular techniques to identify deletion events that remove genomic sequence from the gene of interest, and assessed for phenotypic effects in heterozygous and homozygous mutant Drosophila.

Recently a transgenesis system has been described that may have universal applicability in all eye-bearing animals and which has been proven effective in delivering transgenes to diverse insect species (Berghammer et al., Nature (1999) 402:370-371). This system includes: an artificial promoter active in eye tissue of all animal species, preferably containing three Pax6 binding sites positioned upstream of a TATA box (3xP3; Sheng et al., Genes Devel. (1997) 11:1122-1131); a strong and visually detectable marker gene, such as GFP or other autofluorescent protein genes (Pasher et al., Gene (1992) 111:229-233; U.S. Pat. No 5,491,084); and promiscuous vectors capable of delivering transgenes to a broad range of animal species. Examples of promiscuous vectors include transposon-based vectors derived from Hermes, PiggyBac, or mariner, and vectors based on pantropic VSVc-pseudotyped retroviruses (Burns et al., In Vitro Cell Dev Biol Anim (1996) 32:78-84; Jordan et al., Insect Mol Biol (1998) 7: 215-222; U.S. Pat. No. 5,670,345). Thus, since the same transgenesis system can be used in a variety of phylogenetically diverse animals, comparative functional studies are greatly facilitated, which is especially helpful in evaluating new applications to pest management.

In C. elegans, Tc1 transposable element can be used for directed mutagenesis of a gene of interest. Typically, a Tc1 library is prepared by the methods of Zwaal et al., supra and Plasterk, supra, using a strain in which the Tc1 transposable element is highly mobile and present in a high copy number. The library is screened for Tc1 insertions in the region of interest using PCR with one set of primers specific for Tc1 sequence and one set of gene-specific primers and C. elegans strains that contain Tc1 transposon insertions within the gene of interest are isolated.

In addition to creating loss-of-function phenotypes, transposable elements can be used to incorporate the gene of interest, or mutant or derivative thereof, as an additional gene into any region of an animal's genome resulting in mis-expression (including over-expression) of the gene. A preferred vector designed specifically for misexpression of genes in transgenic Drosophila, is derived from pGMR (Hay et al., Development (1994) 120:2121-2129), is 9 Kb long, and contains: an origin of replication for E. coli; an ampicillin resistance gene; P element transposon 3′ and 5′ ends to mobilize the inserted sequences; a White marker gene; an expression unit comprising the TATA region of hsp70 enhancer and the 3′untranslated region of α-tubulin gene. The expression unit contains a first multiple cloning site (MCS) designed for insertion of an enhancer and a second MCS located 500 bases downstream, designed for the insertion of a gene of interest. As an alternative to transposable elements, homologous recombination or gene targeting techniques can be used to substitute a gene of interest for one or both copies of the animal's homologous gene. The transgene can be under the regulation of either an exogenous or an endogenous promoter element, and be inserted as either a minigene or a large genomic fragment. In one application, gene function can be analyzed by ectopic expression, using, for example, Drosophila (Brand et al., Methods in Cell Biology (1994) 44:635-654) or C. elegans (Mello and Fire, Methods in Cell Biology (1995) 48:451-482).

Examples of well-characterized heterologous promoters that may be used to create the transgenic animals include heat shock promoters/enhancers, which are useful for temperature induced mis-expression. In Drosophila, these include the hsp70 and hsp83 genes, and in C. elegans, include hsp 16-2 and hsp 16-41. Tissue specific promoters/enhancers are also useful, and in Drosophila, include eyeless (Mozer and Benzer, Development (1994) 120:1049-1058), sevenless (Bowtell et al., PNAS (1991) 88(15):6853-6857), and glass-responsive promoters/enhancers (Quiring et al., Science (1994) 265:785-789) which are useful for expression in the eye; and enhancers/promoters derived from the dpp or vestigal genes which are useful for expression in the wing (Staehling-Hampton et al., Cell Growth Differ. (1994) 5(6):585-593; Kim et al., Nature (1996) 382:133-138). Finally, where it is necessary to restrict the activity of dominant active or dominant negative transgenes to regions where the pathway is normally active, it may be useful to use endogenous promoters of genes in the pathway, such as the subject pathway genes.

In C. elegans, examples of useful tissue specific promoters/enhancers include the myo-2 gene promoter, useful for pharyngeal muscle-specific expression; the hlh-1 gene promoter, useful for body-muscle-specific expression; and the gene promoter, useful for touch-neuron-specific gene expression. In a preferred embodiment, gene fusions for directing the mis-expression of subject pathway genes are incorporated into a transformation vector which is injected into nematodes along with a plasmid containing a dominant selectable marker, such as rol-6. Transgenic animals are identified as those exhibiting a roller phenotype, and the transgenic animals are inspected for additional phenotypes of interest created by mis-expression of the subject pathway gene.

In Drosophila, binary control systems that employ exogenous DNA are useful when testing the mis-expression of genes in a wide variety of developmental stage-specific and tissue-specific patterns. Two examples of binary exogenous regulatory systems include the UAS/GAL4 system from yeast (Hay et al., PNAS (1997) 94(10):5195-5200; Ellis et al., Development (1993) 119(3):855-865), and the “Tet system” derived from E. coli (Bello et al., Development (1998) 125:2193-2202). The UAS/GAL4 system is a well-established and powerful method of mis-expression in Drosophila which employs the UAS_{G} upstream regulatory sequence for control of promoters by the yeast GAL4 transcriptional activator protein (Brand and Perrimon, Development (1993) 118(2):401-15). In this approach, transgenic Drosophila, termed “target” lines, are generated where the gene of interest to be mis-expressed is operably fused to an appropriate promoter controlled by UAS_{G}. Other transgenic Drosophila strains, termed “driver” lines, are generated where the GAL4 coding region is operably fused to promoters/enhancers that direct the expression of the GAL4 activator protein in specific tissues, such as the eye, wing, nervous system, gut, or musculature. The gene of interest is not expressed in the target lines for lack of a transcriptional activator to drive transcription from the promoter joined to the gene of interest. However, when the UAS-target line is crossed with a GAL4 driver line, mis-expression of the gene of interest is induced in resulting progeny in a specific pattern that is characteristic for that GAL4 line. The technical simplicity of this approach makes it possible to sample the effects of directed mis-expression of the gene of interest in a wide variety of tissues by generating one transgenic target line with the gene of interest, and crossing that target line with a panel of pre-existing driver lines.

In the “Tet” binary control system, transgenic Drosophila driver lines are generated where the coding region for a tetracycline-controlled transcriptional activator (tTA) is operably fused to promoters/enhancers that direct the expression of tTA in a tissue-specific and/or developmental stage-specific manner. The driver lines are crossed with transgenic Drosophila target lines where the coding region for the gene of interest to be mis-expressed is operably fused to a promoter that possesses a tTA-responsive regulatory element. When the resulting progeny are supplied with food supplemented with a sufficient amount of tetracycline, expression of the gene of interest is blocked. Expression of the gene of interest can be induced at will simply by removal of tetracycline from the food. Also, the level of expression of the gene of interest can be adjusted by varying the level of tetracycline in the food. Thus, the use of the Tet system as a binary control mechanism for mis-expression has the advantage of providing a means to control the amplitude and timing of mis-expression of the gene of interest, in addition to spatial control. Consequently, if a gene of interest (e.g. a subject gene) has lethal or deleterious effects when mis-expressed at an early stage in development, such as the embryonic or larval stages, the function of the gene of interest in the adult can still be assessed by adding tetracycline to the food during early stages of development and removing tetracycline later so as to induce mis-expression only at the adult stage.

Dominant negative mutations, by which the mutation causes a protein to interfere with the normal function of a wild-type copy of the protein, and which can result in loss-of-function or reduced-function phenotypes in the presence of a normal copy of the gene, can be made using known methods (Hershkowitz, Nature (1987) 329:219-222). In the case of active monomeric proteins, overexpression of an inactive form, achieved, for example, by linking the mutant gene to a highly active promoter, can cause competition for natural substrates or ligands sufficient to significantly reduce net activity of the normal protein. Alternatively, changes to active site residues can be made to create a virtually irreversible association with a target.

### Assays for Change in Gene Expression

Various expression analysis techniques may be used to identify genes which are differentially expressed between a cell line or an animal expressing a wild type subject gene compared to another cell line or animal expressing a mutant subject gene. Such expression profiling techniques include differential display, serial analysis of gene expression (SAGE), transcript profiling coupled to a gene database query, nucleic acid array technology, subtractive hybridization, and proteome analysis (e.g. mass-spectrometry and two-dimensional protein gels). Nucleic acid array technology may be used to determine a global (i.e., genome-wide) gene expression pattern in a normal animal for comparison with an animal having a mutation in subject gene. Gene expression profiling can also be used to identify other genes (or proteins) that may have a functional relation to subject (e.g. may participate in a signaling pathway with the subject gene). The genes are identified by detecting changes in their expression levels following mutation, i.e., insertion, deletion or substitution in, or over-expression, under-expression, mis-expression or knock-out, of the subject gene.

### Phenotypes Associated with Subject Pathway Gene Mutations

After isolation of model animals carrying mutated or mis-expressed subject pathway genes or inhibitory RNAs, animals are carefully examined for phenotypes of interest. For analysis of subject pathway genes that have been mutated (i.e. deletions, insertions, and/or point mutations) animal models that are both homozygous and heterozygous for the altered subject pathway gene are analyzed. Examples of specific phenotypes that may be investigated include lethality; sterility; feeding behavior, perturbations in neuromuscular function including alterations in motility, and alterations in sensitivity to pharmaceuticals. Some phenotypes more specific to flies include alterations in: adult behavior such as, flight ability, walking, grooming, phototaxis, mating or egg-laying; alterations in the responses of sensory organs, changes in the morphology, size or number of adult tissues such as, eyes, wings, legs, bristles, antennae, gut, fat body, gonads, and musculature; larval tissues such as mouth parts, cuticles, internal tissues or imaginal discs; or larval behavior such as feeding, molting, crawling, or puparian formation; or developmental defects in any germline or embryonic tissues. Some phenotypes more specific to nematodes include: locomotory, egg laying, chemosensation, male mating, and intestinal expulsion defects. In various cases, single phenotypes or a combination of specific phenotypes in model organisms might point to specific genes or a specific pathway of genes, which facilitate the cloning process.

Genomic sequences containing a subject pathway gene can be used to confirm whether an existing mutant insect or worm line corresponds to a mutation in one or more subject pathway genes, by rescuing the mutant phenotype. Briefly, a genomic fragment containing the subject pathway gene of interest and potential flanking regulatory regions can be subcloned into any appropriate insect (such as Drosophila) or worm (such as C. elegans) transformation vector, and injected into the animals. For Drosophila, an appropriate helper plasmid is used in the injections to supply transposase for transposon-based vectors. Resulting germline transformants are crossed for complementation testing to an existing or newly created panel of Drosophila or C. elegans lines whose mutations have been mapped to the vicinity of the gene of interest (Fly Pushing: The Theory and Practice of Drosophila Genetics, supra; and Caenorhabditis elegans: Modem Biological Analysis of an Organism (1995), Epstein and Shakes, eds.). If a mutant line is discovered to be rescued by this genomic fragment, as judged by complementation of the mutant phenotype, then the mutant line likely harbors a mutation in the subject pathway gene. This prediction can be further confirmed by sequencing the subject pathway gene from the mutant line to identify the lesion in the subject pathway gene.

### Identification of Genes that Modify Subject Genes

The characterization of new phenotypes created by mutations or misexpression in subject genes enables one to test for genetic interactions between subject genes and other genes that may participate in the same, related, or interacting genetic or biochemical pathway(s). Individual genes can be used as starting points in large-scale genetic modifier screens as described in more detail below. Alternatively, RNAi methods can be used to simulate loss-of-function mutations in the genes being analyzed. It is of particular interest to investigate whether there are any interactions of subject genes with other well-characterized genes, particularly genes involved in metabolism.

### Genetic Modifier Screens

A genetic modifier screen using invertebrate model organisms is a particularly preferred method for identifying genes that interact with subject genes, because large numbers of animals can be systematically screened making it more possible that interacting genes will be identified. In Drosophila, a screen of up to about 10,000 animals is considered to be a pilot-scale screen. Moderate-scale screens usually employ about 10,000 to about 50,000 flies, and large-scale screens employ greater than about 50,000 flies. In a genetic modifier screen, animals having a mutant phenotype due to a mutation in or misexpression of one or more subject genes are further mutagenized, for example by chemical mutagenesis or transposon mutagenesis.

The procedures involved in typical Drosophila genetic modifier screens are well-known in the art (Wolfner and Goldberg, Methods in Cell Biology (1994) 44:33-80; and Karim et al., Genetics (1996)143:315-329). The procedures used differ depending upon the precise nature of the mutant allele being modified. If the mutant allele is genetically recessive, as is commonly the situation for a loss-of-function allele, then most typically males, or in some cases females, which carry one copy of the mutant allele are exposed to an effective mutagen, such as EMS, MMS, ENU, triethylamine, diepoxyalkanes, ICR-170, formaldehyde, X-rays, gamma rays, or ultraviolet radiation. The mutagenized animals are crossed to animals of the opposite sex that also carry the mutant allele to be modified. In the case where the mutant allele being modified is genetically dominant, as is commonly the situation for ectopically expressed genes, wild type males are mutagenized and crossed to females carrying the mutant allele to be modified.

The progeny of the mutagenized and crossed flies that exhibit either enhancement or suppression of the original phenotype are presumed to have mutations in other genes, called “modifier genes”, that participate in the same phenotype-generating pathway. These progeny are immediately crossed to adults containing balancer chromosomes and used as founders of a stable genetic line. In addition, progeny of the founder adult are retested under the original screening conditions to ensure stability and reproducibility of the phenotype. Additional secondary screens may be employed, as appropriate, to confirm the suitability of each new modifier mutant line for further analysis.

Standard techniques used for the mapping of modifiers that come from a genetic screen in Drosophila include meiotic mapping with visible or molecular genetic markers; male-specific recombination mapping relative to P-element insertions; complementation analysis with deficiencies, duplications, and lethal P-element insertions; and cytological analysis of chromosomal aberrations (Fly Pushing: Theory and Practice of Drosophila Genetics, supra; Drosophila: A Laboratory Handbook, supra). Genes corresponding to modifier mutations that fail to complement a lethal P-element may be cloned by plasmid rescue of the genomic sequence surrounding that P-element. Alternatively, modifier genes may be mapped by phenotype rescue and positional cloning (Sambrook et al., supra).

Newly identified modifier mutations can be tested directly for interaction with other genes of interest known to be involved or implicated with subject genes using methods described above. Also, the new modifier mutations can be tested for interactions with genes in other pathways that are not believed to be related to metabolism (e.g. nanos in Drosophila). New modifier mutations that exhibit specific genetic interactions with other genes implicated in metabolism, but not interactions with genes in unrelated pathways, are of particular interest.

The modifier mutations may also be used to identify “complementation groups”. Two modifier mutations are considered to fall within the same complementation group if animals carrying both mutations in trans exhibit essentially the same phenotype as animals that are homozygous for each mutation individually and, generally are lethal when in trans to each other (Fly Pushing: The Theory and Practice of Drosophila Genetics, supra). Generally, individual complementation groups defined in this way correspond to individual genes.

When subject modifier genes are identified, homologous genes in other species can be isolated using procedures based on cross-hybridization with modifier gene DNA probes, PCR-based strategies with primer sequences derived from the modifier genes, and/or computer searches of sequence databases. For therapeutic applications related to the function of subject genes, human and rodent homologs of the modifier genes are of particular interest.

Although the above-described Drosophila genetic modifier screens are quite powerful and sensitive, some genes that interact with subject genes may be missed in this approach, particularly if there is functional redundancy of those genes. This is because the vast majority of the mutations generated in the standard mutagenesis methods will be loss-of-function mutations, whereas gain-of-function mutations that could reveal genes with functional redundancy will be relatively rare. Another method of genetic screening in Drosophila has been developed that focuses specifically on systematic gain-of-function genetic screens (Rorth et al., Development (1998) 125:1049-1057). This method is based on a modular mis-expression system utilizing components of the GAL4/UAS system (described above) where a modified P element, termed an “enhanced P” (EP) element, is genetically engineered to contain a GAL4-responsive UAS element and promoter. Any other transposons can also be used for this system. The resulting transposon is used to randomly tag genes by insertional mutagenesis (similar to the method of P element mutagenesis described above). Thousands of transgenic Drosophila strains, termed EP lines, can be generated, each containing a specific UAS-tagged gene. This approach takes advantage of the preference of P elements to insert at the 5′-ends of genes. Consequently, many of the genes that are tagged by insertion of EP elements become operably fused to a GAL4-regulated promoter, and increased expression or mis-expression of the randomly tagged gene can be induced by crossing in a GAL4 driver gene.

Systematic gain-of-function genetic screens for modifiers of phenotypes induced by mutation or mis-expression of a subject gene can be performed by crossing several thousand Drosophila EP lines individually into a genetic background containing a mutant or mis-expressed subject gene, and further containing an appropriate GAL4 driver transgene. It is also possible to remobilize the EP elements to obtain novel insertions. The progeny of these crosses are then analyzed for enhancement or suppression of the original mutant phenotype as described above. Those identified as having mutations that interact with the subject gene can be tested further to verify the reproducibility and specificity of this genetic interaction. EP insertions that demonstrate a specific genetic interaction with a mutant or mis-expressed subject gene, have a physically tagged new gene which can be identified and sequenced using PCR or hybridization screening methods, allowing the isolation of the genomic DNA adjacent to the position of the EP element insertion.

### EXAMPLES

The following examples describe the isolation and cloning of the nucleic acid sequence of SEQ ID NOs:1, 3, 5, 7, and 9 and how these sequences, and derivatives and fragments thereof, as well as other subject pathway nucleic acids and gene products can be used for genetic studies to elucidate mechanisms of the subject pathway as well as the discovery of potential pharmaceutical agents that interact with the pathway.

These Examples are provided merely as illustrative of various aspects of the invention and should not be construed to limit the invention in any way.

### Example 1

### Preparation of Drosophila CDNA Library

A Drosophila expressed sequence tag (EST) cDNA library was prepared as follows. Tissue from mixed stage embryos (0-20 hour), imaginal disks and adult fly heads were collected and total RNA was prepared. Mitochondrial rRNA was removed from the total RNA by hybridization with biotinylated rRNA specific oligonucleotides and the resulting RNA was selected for polyadenylated mRNA. The resulting material was then used to construct a random primed library. First strand cDNA synthesis was primed using a six nucleotide random primer. The first strand cDNA was then tailed with terminal transferase to add approximately 15 dGTP molecules. The second strand was primed using a primer which contained a Not1 site followed by a 13 nucleotide C-tail to hybridize to the G-tailed first strand cDNA. The double stranded cDNA was ligated with BstX1 adaptors and digested with Not1. The cDNA was then fractionated by size by electrophoresis on an agarose gel and the cDNA greater than 700 bp was purified. The cDNA was ligated with Not1, BstX1 digested pCDNA−sk+vector (a derivative of pBluescript, Stratagene) and used to transform E. coli (XL1blue). The final complexity of the library was 6×10⁶ independent clones.

The cDNA library was normalized using a modification of the method described by Bonaldo et al. (Genome Research (1996) 6:791-806). Biotinylated driver was prepared from the cDNA by PCR amplification of the inserts and allowed to hybridize with single stranded plasmids of the same library. The resulting double-stranded forms were removed using strepavidin magnetic beads, the remaining single stranded plasmids were converted to double stranded molecules using Sequenase (Amersham, Arlington Hills, Ill.), and the plasmid DNA stored at −20° C. prior to transformation. Aliquots of the normalized plasmid library were used to transform E. coli (XL1blue or DH10B), plated at moderate density, and the colonies picked into a 384-well master plate containing bacterial growth media using a Qbot robot (Genetix, Christchurch, UK). The clones were allowed to grow for 24 hours at 37° C. then the master plates were frozen at −80° C. for storage. The total number of colonies picked for sequencing from the normalized library was 240,000. The master plates were used to inoculate media for growth and preparation of DNA for use as template in sequencing reactions. The reactions were primarily carried out with primer that initiated at the 5′ end of the cDNA inserts. However, a minor percentage of the clones were also sequenced from the 3′ end. Clones were selected for 3′ end sequencing based on either further biological interest or the selection of clones that could extend assemblies of contiguous sequences (“contigs”) as discussed below. DNA sequencing was carried out using ABI377 automated sequencers and used either ABI FS, dirhodamine or BigDye chemistries (Applied Biosystems, Inc., Foster City, Calif.).

Analysis of sequences were done as follows: the traces generated by the automated sequencers were base-called using the program “Phred” (Gordon, Genome Res. (1998) 8:195-202), which also assigned quality values to each base. The resulting sequences were trimmed for quality in view of the assigned scores. Vector sequences were also removed. Each sequence was compared to all other fly EST sequences using the BLAST program and a filter to identify regions of near 100% identity. Sequences with potential overlap were then assembled into contigs using the programs “Phrap”, “Phred” and “Consed” (Phil Green, University of Washington, Seattle, Wash.; http://bozeman.mbt.washington.edu/phrap.docs/phrap.html). The resulting assemblies were then compared to existing public databases and homology to known proteins was then used to direct translation of the consensus sequence. Where no BLAST homology was available, the statistically most likely translation based on codon and hexanucleotide preference was used. The Pfam (Bateman et al., Nucleic Acids Res. (1999) 27:260-262) and Prosite (Hofmann et al., Nucleic Acids Res. (1999) 27(1):215-219) collections of protein domains were used to identify motifs in the resulting translations. The contig sequences were archived in an Oracle-based relational database (FlyTag™, Exelixis, Inc., South San Francisco, Calif.).

### Example 2

### Cloning of Nucleic Acid Sequences

Unless otherwise noted, the PCR conditions used for cloning each subject nucleic acid sequence was as follows: A denaturation step of 94° C., 5 min; followed by 35 cycles of: 94° C. 1 min, 55° C. 1 min 72° C. 1 min; then, a final extension at 72° C. 10 min.

All DNA sequencing reactions were performed using standard protocols for the BigDye sequencing reagents (Applied Biosystems, Inc.) and products were analyzed using ABI 377 DNA sequencers. Trace data obtained from the ABI 377 DNA sequencers was analyzed and assembled into contigs using the Phred-Phrap programs.

Well-separated, single colonies were streaked on a plate and end-sequenced to verify the clones. Single colonies were picked and the enclosed plasmid DNA was purified using Qiagen REAL Preps (Qiagen, Inc., Valencia, Calif.). Samples were then digested with appropriate enzymes to excise insert from vector and determine size, for example the vector pOT2, (www.fruitfly.org/EST/pOT2vector.htm1) and can be excised with Xho1/EcoRI; or pBluescript (Stratagene) and can be excised with BssH II. Clones were then sequenced using a combination of primer walking and in vitro transposon tagging strategies.

For primer walking, primers were designed to the known DNA sequences in the clones, using the Primer-3 software (Steve Rozen, Helen J. Skaletsky (1998) Primer3. Code available at http://www-genome.wi.mit.edu/genome_software/other/primer3.html.). These primers were then used in sequencing reactions to extend the sequence until the full sequence of the insert was determined.

The GPS-1 Genome Priming System in vitro transposon kit (New England Biolabs, Inc., Beverly, Mass.) was used for transposon-based sequencing, following manufacturer's protocols. Briefly, multiple DNA templates with randomly interspersed primer-binding sites were generated. These clones were prepared by picking 24 colonies/clone into a Qiagen REAL Prep to purify DNA and sequenced by using supplied primers to perform bidirectional sequencing from both ends of transposon insertion.

Sequences were then assembled using Phred/Phrap and analyzed using Consed. Ambiguities in the sequence were resolved by resequencing several clones.

For dmAPS, this effort resulted in a contiguous nucleotide sequence of 2911 bases in length, encompassing an open reading frame (ORF) of 1824 nucleotides encoding a predicted protein of 608 amino acids. The ORF extends from base 447-2373 of SEQ ID NO:1.

For dmCYP, this effort resulted in a contiguous nucleotide sequence of 1683 bases in length, encompassing an open reading frame (ORF) of 1548 nucleotides encoding a predicted protein of 516 amino acids. The ORF extends from base 22-1570 of SEQ ID NO:3.

For dmIGF, this effort resulted in a contiguous nucleotide sequence of 703 bases in length, encompassing an open reading frame (ORF) of 413 nucleotides encoding a predicted protein of 137 amino acids. The ORF extends from base 78-488 of SEQ ID NO:5.

For dmSHIP2A, this effort resulted in a contiguous nucleotide sequence of 1813 nucleotides in length, encompassing an open reading frame (ORF) of 1071 nucleotides encoding a predicted protein of 357 amino acids. The ORF extends from base 235-1308 of SEQ ID NO:7.

For dmSHIP2B, this effort resulted in a contiguous nucleotide sequence of 4175 bases in length, encompassing an open reading frame (ORF) of 3342 nucleotides encoding a predicted protein of 1114 amino acids. The ORF extends from base 214-3558 of SEQ ID NO:9.

### Example 3

### Analysis of dmAPS Nucleic Acid Sequences

Upon completion of cloning, the sequences were analyzed using the Pfam and Prosite programs. Pfam predicted a Pleckstrin Homology (PH) domain (PF00169) at amino acids 285-307 (nucleotides 1301-1367), and an Src Homology 2 (SH2) domain (PF00017) at amino acids 442-519 (nucleotides 1771-2003).

Nucleotide and amino acid sequences for the dmAPS nucleic acid sequences and their encoded proteins were searched against all available nucleotide and amino acid sequences in the public databases, using BLAST (Altschul et al., supra). Table 1 below summarizes the results. The 5 most similar sequences are listed.

TABLE 1

| GI# | DESCRIPTION |
|---|---|
| DNA | |
| BLAST | |
| 6633921 = | Drosophila melanogaster chromosome 3 clone |
| AC008209 | BACR06K17 (D771) RPCI-98 06.K.17 map 96F-96F |
| | strain y; cn bw sp, ***SEQUENCING |
| | TIN PROGRESS ***, 70 unordered |
| | pieces |
| 6446423 = | Drosophila melanogaster Lnk-like mRNA sequence |
| AF101158 | |
| 3101723 = | Drosophila melanogaster cDNA clone LD26138 5prime, |
| AA942100 | mRNA sequence |
| 6446424 = | Drosophila melanogaster Lnk-like protein mRNA, |
| AF101159 | partial cds |
| 5615181 = | Drosophila melanogaster genome survey sequence T7 |
| AL103570 | end of BAC BACN11N09 of DrosBAC library from |
| | Drosophila melanogaster (fruit fly), genomic survey |
| | sequence |
| PROTEIN | |
| BLAST | |
| 6446425 = | Lnk-like protein [Drosophila melanogaster] |
| AAF08615 | |
| 5305448 = | SH2-B PH domain containing signaling mediator 1 |
| AAD41655 | gamma isoform [Mus musculus] |
| 2772908 = | Pro-rich, PH, SH2 domain-containing signaling |
| AAC33414 | mediator [Mus musculus] |
| 3766234 = | APS protein [Rattus norvegicus] |
| AAC64408 | |
| 2447036 = | APS [Homo sapiens] |
| BAA22514 | |

The closest homolog predicted by BLAST analysis is a Drosophila Lnk-like protein, which is identical to the region of 405-606 of SEQ ID NO:2. The BLAST analysis also revealed several other proteins of the APS family which share significant amino acid homology with dmAPS.

APS is an adapter protein with pleckstrin homology (PH) and src homology-2 (SH2) domains. dmAPS protein is predicted to be 608 amino acids in length. The SH2 domain is a small protein region that is found in a wide variety of proteins and acts as a phosphate binding loop. The SH2 domain usually contains a highly conserved FLVRES sequence involved in phosphate binding. The dmAPS contains the very similar FLVRQS sequence.

BLAST results for the dmAPS amino acid sequence indicate 200 amino acid residues as the shortest stretch of contiguous amino acids that is novel with respect to published sequences and 200 amino acids as the shortest stretch of contiguous amino acids for which there are no sequences contained within public database sharing 100% sequence similarity.

### Example 4

Analysis of dmCYP Nucleic Acid Sequences

Upon completion of cloning, the sequences were analyzed using the Pfam and Prosite programs. One transmembrane domain was predicted at amino acids 1-17, corresponding to nucleotides 22-72. Additionally, a Cytochrome P450 domain was recognized (PF00067) at amino acids 35-505, corresponding to nucleotides 124-1526.

Nucleotide and amino acid sequences for the dmCYP nucleic acid sequence and encoded protein were searched against all available nucleotide and amino acid sequences in the public databases, using BLAST (Altschul et al., supra). Table 1 below summarizes the results. The 5 most similar sequences are listed.

TABLE 2

| GI# | DESCRIPTION |
|---|---|
| DNA | |
| BLAST | |
| 4529969 = | Drosophila melanogaster, chromosome 2R, region 44C3- |
| AC005451 | 44D2, P1 clone DS08332, complete sequence |
| 6664495 = | Drosophila melanogaster, *** SEQUENCING IN |
| AC020402 | PROGRESS ***, in ordered pieces |
| 1480636 = | Drosophila melanogaster cytochrome P450 (CYP4E2) |
| U56957 | mRNA, complete cds |
| 2776443 = | Drosophila melanogaster cDNA clone LD02646 5prime, |
| AA202364 | mRNA sequence |
| 6466503 = | Drosophila melanogaster chromosome 2 clone DS00150 |
| AC005415 | (D265) map 51E9-51F2 strain y; cn bw sp, *** |
| | SEQUENCING IN PROGRESS ***, 86 unordered pieces |
| PROTEIN | |
| BLAST | |
| 2674280 = | microsomal cytochrome P450 [Drosophila mettleri] |
| AAC27534 | |
| 1480637 = | cytochrome P450 [Drosophila melanogaster] |
| AAC47424 | |
| 2133647 = | cytochrome P450, Cyp4e2 - fruit fly (Drosophila |
| JC5236 | melanogaster) |
| 2351797 = | cytochrome P450 monooxygenase CYP4D10 [Drosophila |
| AAB68664 | mettleri] |
| 2431964 = | cytochrome P450 [Drosophila simulans] |
| AAB71182 | |

The closest homolog predicted by BLAST analysis is a cytochrome p450 from Drosophila with 33% identity and 53% homology with dmCYP. BLAST analysis of the amino acid sequence reveals modest identity (˜30%) to a number of cytochromes, almost exclusively from the CYP4 family. These include, CYP4W1 (33%), CYP4E2 (37%), and CYP4D10 (35%). BLAST results for the dmCYP amino acid sequence indicate 14 amino acid residues as the shortest stretch of contiguous amino acids that is novel with respect to published sequences and 16 amino acids as the shortest stretch of contiguous amino acids for which there are no sequences contained within public database sharing 100% sequence similarity.

### Example 5

### Analysis of dmIGF Nucleic Acid Sequences

Upon completion of cloning, the sequences were analyzed using the PSORT (Nakai K., and Horton P., Trends Biochem Sci, 1999, 24:34-6) and Prosite (Bairoch, A. PROSITE: A DICTIONARY OF PROTEIN SITES AND PATTERNS USER MANUAL Release 14.0, November 1997) programs. PSORT predicted an amino-terminal membrane-spanning domain, at amino acids 5-21. Prosite predicted an insulin family motif, from amino acids 118-132 (nucleotide residues 429-473). BLAST analysis reveals significant homologies to members of the insulin family. These family members contain conserved cysteines, which participate in disulfide bonds. The most closely related sequences are the insulin-like growth factors (IGF), primarily of the IGFII sub-family.

Nucleotide and amino acid sequences for the dmIGF nucleic acid sequences and their encoded proteins were searched against all available nucleotide and amino acid sequences in the public databases, using BLAST (Altschul et al., supra). Table 1 below summarizes the results. The 5 most similar sequences are listed.

TABLE 3

| GI# | DESCRIPTION |
|---|---|
| DNA | |
| BLAST | |
| 6436959 = | Drosophila melanogaster, *** SEQUENCING IN |
| AC014376.1 | PROGRESS ***, in ordered pieces |
| 3834268 = | Drosophila melanogaster cDNA clone LD16278 3prime |
| AA441371 | |
| WO | Homo sapiens adult placenta clone DA136_11 3'region |
| 9814576-A2, | |
| Claim 45 | |
| 6727776 = | 5910 MARC 1PIG Sus scrofa cDNA 5' |
| AW311906 | |
| 2153249 = | 5prime LD Drosophila Embryo Drosophila |
| AA441371 | melanogaster cDNA clone |
| PROTEIN | |
| BLAST | |
| 2133793 = | insulin-like growth factor II precursor - spiny dogfish |
| S66484 | |
| 902733 = | insulin-like growth factor II [Squalus acanthias] |
| CAA90413 | |
| (Z50082) | |
| 217244 = | bombyxin B-9 precursor [Bombyx mori] |
| BAA00681 | |
| (D00785) | |
| EP128733-A | Fusion protein of insulin-like growth factor 1 and yeast |
| | invertase |
| EP128733-A | Human insulin-like growth factor II |

The closest homolog predicted by BLAST analysis is an insulin like growth factor II (IGFII) from spiny dogfish with 58% identity and 74% similarity to dmIGF. The BLAST analysis with dmIGF protein also revealed several other proteins of the insulin superfamily, from both vertebrate and invertebrate species, which share significant amino acid homology, primarily within the insulin family motif.

BLAST results for the dmIGF amino acid sequence indicate 5 amino acid residues as the shortest stretch of contiguous amino acids that is novel with respect to published sequences and 10 amino acids as the shortest stretch of contiguous amino acids for which there are no sequences contained within public database sharing 100% sequence similarity.

### Example 6

### Analysis of dmSHIP2A Nucleic Acid Sequences

Upon completion of cloning, the sequences were analyzed using the Pfam and Prosite programs. Analysis of dmSHIP2A reveals two putative transmembrane domains at amino acids 1-17 and 336-352, corresponding to nucleotides 235-284 and 1240-1290. Pfam predicted an inositol polyphosphate phosphatase domain (PF00783) at amino acids 8-316, corresponding to nucleotides 256-1182.

Nucleotide and amino acid sequences for the dmSHIP2A nucleic acid sequence and its encoded protein were searched against all available nucleotide and amino acid sequences in the public databases, using BLAST (Altschul et al., supra). Table 1 below summarizes the results. The 5 most similar sequences are listed.

TABLE 4

| | |
|---|---|
| GI# | DESCRIPTION |

| | |
|---|---|
| DNA | |
| BLAST | |
| 4019173 = | Drosophila melanogaster, chromosome 2R, region 53E1- |
| AC004335 | 53F1, P1 clone DS03108, complete sequence |
| 2790386 = | Drosophila melanogaster cDNA clone LD09367 5prime, |
| AA390465 | mRNA sequence |
| 1704635 = | Drosophila melanogaster cDNA clone CK01299 5prime, |
| AA141028 | mRNA sequence |
| 3111911 = | Drosophila melanogaster cDNA clone LD29153 5prime, |
| AA952098 | mRNA sequence |
| 1704636 = | Drosophila melanogaster cDNA clone CK01299 3prime, |
| AA141029 | mRNA sequence |
| PROTEIN | |
| BLAST | |
| 4314432 = | similar to phosphatidylinositol (4,5)bisphosphate 5- |
| AAD15618 | phosphatase; match to PID:g1399105 [Homo sapiens] |
| 2121246 = | putative phosphoinositide 5-phosphatase type II [Mus |
| AAC53265 | musculus] |
| 2121241 = | putative phosphoinositide 5-phosphatase type II; C62 [Mus |
| AAC60757 | musculus] |
| 1019103 = | inositol polyphosphate 5-phosphatase [Homo sapiens] |
| AAA79207 | |
| 3241987 = | synaptojanin 2 isoform delta [Mus musculus] |
| AAC40142 | |

The closest homolog predicted by BLAST analysis is a protein similar to phosphatidylinositol (4,5)bisphosphate 5-phosphatase from human, with 39% identity and 61% homology.

dmSHIP2A sequence does not contain the proline-rich C-terminus that normally constitutes the putative SH3-binding domain of SHIP2. The consensus sequence for this domain varies as either ‘PXXP’ (Wisniewski et al., Blood (1999) 93(8):2707-2720) or ‘PXXPXR’ (Ishihara et al., Biochem. Biophys. Res. Comm. (1999) 260:265-272). dmSHIP2A sequence satisfies the ‘PXXP’ (P335, A336, T337, P338) requirement, but not the ‘PXXPXR’ consensus. In addition, the proline-rich C-terminus of rat and human SHIP2 is characterized by an occurrence of about 55 prolines in a sequence of about 250 amino acids (˜20%). This is clearly absent from dmAHIP2A. Furthermore, dmSHIP2A lacks any of the phosphotyrosine binding consensus sequences described by ‘NPXY’ as found in both SHIP1 and SHIP2. It does, however, contain two segments resembling rat SHIP2 that constitute the two conserved 5′-phosphatase motifs (Ishihara et al., supra).

BLAST results for the dmSHIP2A amino acid sequence indicate 10 amino acid residues as the shortest stretch of contiguous amino acids that is novel with respect to published sequences and 18 amino acids as the shortest stretch of contiguous amino acids for which there are no sequences contained within public database sharing 100% sequence similarity.

### Example 7

### Analysis of dmSHIP2B Nucleic Acid Sequences

Upon completion of cloning, the sequences were analyzed using the Pfam and Prosite programs. The following structural domains were predicted: a possible cleavage site was predicted between amino acids 44 and 45 (nucleotides 345 and 348) and a putative transmembrane domain at amino acids 76-92 (nucleotides 439-489. Pfam predicted an inositol polyphosphate phosphatase (PF00783), at amino acids 536-869 (nucleotides 1819-2820).

Nucleotide and amino acid sequences for each of the dmSHIP2B nucleic acid sequence and its encoded protein were searched against all available nucleotide and amino acid sequences in the public databases, using BLAST (Altschul et al., supra). Table 1 below summarizes the results. The 5 most similar sequences are listed.

TABLE 5

| GI# | DESCRIPTION |
|---|---|
| DNA | |
| BLAST | |
| 3006207 = | Drosophila melanogaster (P1 DS00642 (D59)) DNA |
| AC004365 | sequence, complete sequence |
| 1931196 = | Drosophila melanogaster (subclone 2_c8 from P1 |
| AC000782 | DS00642 (D59)) DNA sequence, complete sequence. |
| 1931198 = | Drosophila melanogaster (subclone 2_b1 from P1 |
| AC000780 | DS00642 (D59)) DNA sequence, complete sequence |
| 1931201 = | Drosophila melanogaster (subclone 1_d12 from P1 |
| AC000777 | DS00642 (D59)) DNA sequence, complete sequence. |
| 4937202 = | Drosophila melanogaster genome survey sequence T7 |
| AL056433 | end of BAC #BACR22E08 of RPCI-98 library |
| PROTEIN | |
| BLAST | |
| 2702321 = | synaptojanin [Homo sapiens] |
| AAC51921 | |
| 2702323 = | synaptojanin [Homo sapiens] |
| AAC51922 | |
| 1586823 = | synaptojanin |
| 2204390A | |
| 1166575 = | synaptojanin [Rattus norvegicus] |
| AAB60525 | |
| 2285875 = | synaptojanin [Bos taurus] |
| BAA21652 | |

BLAST results indicate the amino acid sequence of dmSHIP2B bears ˜50% identity to synaptojanins from various species including rat and human. The proline-rich C-terminus of rat and human SHIP2 is characterized by an occurrence of about 55 prolines in a sequence of about 250 amino acids (˜20%). dmSHIP2B sequence contains a comparable number of prolines in the C-terminus. In addition, the SH3-binding consensus sequence ‘PXXPXR’ (Ishihara, et al., supra) is found in the C-terminus at least once: 1007-PELPQR-1112. However, using the ‘PXXP’ SH3-binding consensus sequence (Wisniewski et al., Blood (1999) 93(8):2707-2720), there are 14 unique occurrences of ‘PXXP’ in the C-terminus, suggesting the possibility of numerous SH3-binding domains.

Interestingly, the phosphotyrosine binding consensus sequences found in SHIP1 and SHIP2 and defined by ‘NPXY’ are not found in dmSHIP2B.

BLAST results for the dmSHIP2B amino acid sequence indicate 20 amino acid residues as the shortest stretch of contiguous amino acids that is novel with respect to published sequences and 38 amino acids as the shortest stretch of contiguous amino acids for which there are no sequences contained within public database sharing 100% sequence similarity.

### Example 8

### In Vitro Interaction Studies with dmAPS

GST fusion proteins are generated by introducing the APS cDNA fragment corresponding to the SH2 domain (amino acids 442-519) into the pGEX5X expression plasmid (Amersham, Piscataway, N.J.). After transformation of DH5, induction with 1 mM isopropyl-1-thio-D-galactopyranoside, cell collection, and lysis by sonication, the proteins are purified using immobilized glutathione-agarose beads. Serum-starved cultured CHO-IR cells are stimulated with 100 nM insulin for 0, 5, 15, or 30 min at 37° C., washed twice with ice-cold 1×PBS, and solubilized with lysis buffer (1×PBS supplemented with 1% Nonidet P-40, 1 mM dithiothreitol, 1 mM phenylmethylsulfonyl fluoride, 1 μg/ml each of aprotinin, leupeptin, and pepstatin, 1 mM sodium vanadate, 10 mM sodium fluoride, 10 mM sodium pyrophosphate). The samples are homogenized and clarified by centrifugation, and incubated (500 μg of total protein/reaction) for 2 hr at 4° C. with 3-5 μg of immobilized GST fusion protein in presence or absence of compounds. After extensive washing with ice-cold HNTG buffer (10 mM HEPES, pH 7.5, 150 mM NaCl, 1% Triton X-100, 10% glycerol), the proteins co-associating with the GST fusion proteins are separated by SDS-polyacrylamide gel electrophoresis, transferred to PVDF membrane (Amersham), and immunoblotted with either anti-phosphotyrosine antibody 4G10 or antibodies against the subunit of the insulin receptor.

### Example 9

### Cytochrome P450 Assay for dCYP Activity, 96-well Format

Test compounds are serially diluted in DMSO to yield a final concentration range of 100, 33.3, 10, 3.3, and 1.0 μM (final DMSO 1%). 100 μL NADPH regeneration system in 100 mM potassium phosphate (pH 7.4) containing 1.3 mM NADP⁺, 3.3 mM Glucose-6-Phosphate, 3.3 mM Magnesium Chloride, and 0.4 U/mL Glucose-6-Phosphate Dehydrogenase is added to the plated compounds. Another solution in 100 mM potassium phosphate (pH 7.4) containing 5-10 pmol of dmCYP, 1 pmol of dmCYP reductase, and fluorescent substrate probe is prepared and immediately dispensed in 100 μL aliquots to the plate containing test compounds and NADPH regeneration system. The plate is incubated at 37° C. for 1 hour and fluorescence read at the appropriate wavelengths suitable for the specific substrate probe used. Each result is compared to a sample on the plate containing no test compound in order to calculate a percent inhibition. The results yield an IC50, the concentration at which test compound inhibits 50% of the total activity. Commercially available substrate probes (available from Gentest Corporation, Woburn, Mass.) include: 7-Benzyloxyquinoline, 7-Benzyloxy-4-(trifluoromethyl)-coumarin, 3-Cyano-7-ethoxycoumarin, 3-Cyano-7-methoxycoumarin, 7-Methoxy-4-(trifluoromethyl)-coumarin, and resorufin esters.

### Example 10

### dmIGF Mitosenic Activity Assay

cDNAs encoding dmIGF are cloned into expression vectors and transfected into cells, and the recombinant dmIGF protein is purified. A cell proliferation assay is performed essentially as described by Marcos and Congote (Biochemistry Journal [1997] 326, 407-413). A Drosophila S2 cell line that does not require serum for viability is maintained at 25 ° C. in Schneider's Drosophila medium, supplemented with 10% fetal bovine serum. Sub-confluent cells are starved overnight, and, after 16 hour supplemented with the media alone or with media containing the purified dmIGF protein. Proliferation of cells is assayed after 48 hours, by the addition of an Alamar Blue solution containing 1.5 μCi of [³H]thymidine. The absorbances of control and experimental samples are determined after 4 h of incubation at 25°. An aliquot from each sample is further processed for determination of thymidine incorporation.

### Example 11

### Effect of Compounds on dmSHIP2A and dmSHIP2Phosphatase Activity

DmSHIP2 constructs may be transfected into cells. Cell extracts are then prepared to assess the activity of phosphatase. Briefly, a total of 15,000 cpm/sample (approximately 60 μM) of [3-³²P]PtIns(3,4,5)P3 (substrate) is resuspended in 100 mM Tris-HCl, pH 7.5, and 1% cholate. The reaction is started by adding 10 μl of enzyme, 5 mM MgCl2, 0.5 mM EGTA, and 0.5% cholate, in presence or absence of 10 μM of compound of interest, for 5 minutes. PtIns(3,4,5)P3 and PtIns(3,4)P2 are separated by thin layer chromatography using 1-propanol/2M acetic acid (1:1). The corresponding spots are also analyzed by phosphorimager and autoradiography.

What is claimed is:

1. An isolated nucleic acid molecule comprising a nucleic acid sequence selected from the group consisting of:
(a) a nucleic acid sequence that encodes a polypeptide comprising at least 70% sequence similarity with any of SEQ ID NOs:2, 4, 6, 8, or 10 and
(b) the complement of the nucleic acid sequence of (a).

2. The isolated nucleic acid molecule of claim 1 wherein said nucleic acid sequence encodes at least one dmAPS functional domain selected from the group consisting of an SH2 domain and a pleckstrin homology domain.

3. The isolated nucleic acid molecule of claim 1 wherein said nucleic acid sequence encodes an inositol polyphosphatase domain.

4. The isolated nucleic acid molecule of claim 1 wherein said nucleic acid sequence encodes an amino acid sequence selected from the group consisting of SEQ ID NOs:2, 4, 6, 8, and 10.

5. A vector comprising the nucleic acid molecule of claim 1.

6. A host cell comprising the vector of claim 5.

7. A process for producing a protein implicated in metabolism comprising culturing the host cell of claim 6 under conditions suitable for expression of said protein and recovering said protein.

8. A purified polypeptide comprising an amino acid sequence sharing at least 80% sequence similarity with an amino acid sequence selected from the group consisting of SEQ ID NOs:2, 4, 6, 8, and 10.

9. A method for detecting a candidate compound that interacts with a protein implicated in metabolism comprising contacting said protein or fragment thereof with one or more candidate molecules, and detecting any interaction between said candidate molecule and said protein, wherein the amino acid sequence of said protein has at least 80% sequence similarity with a sequence selected from the group consisting of SEQ ID NOs:2, 4, 6, 8, and 10.

10. The method of claim 9 wherein said candidate molecule is a putative pharmaceutical agent.

11. The method of claim 9 wherein said contacting comprises administering said candidate compound to cultured host cells that have been genetically engineered to express said protein.

12. The method of claim 9 wherein said contacting comprises administering said candidate compound to a metazoan invertebrate organism that has been genetically engineered to express said protein.

13. The method of claim 12 wherein said organism is an insect or worm.

14. A first animal that is an insect or a worm that has been genetically modified to express or mis-express a protein implicated in metabolism, or the progeny of said animal that has inherited said protein expression or mis-expression, wherein said protein has at least 80% sequence similarity with a sequence selected from the group consisting of SEQ ID NO:2, 4, 6, 8, and 10.

15. A method for studying proteins implicated in metabolism comprising detecting a phenotype caused by the expression or mis-expression of said protein in the first animal of claim 14.

16. The method of claim 15 additionally comprising observing a second animal having the same genetic modification as said first animal which causes said expression or mis-expression, and wherein said second animal additionally comprises a mutation in a gene of interest, wherein differences, if any, between the phenotype of said first animal and the phenotype of said second animal identifies the gene of interest as capable of modifying the function of the protein implicated in metabolism.

17. The method of claim 15 additionally comprising administering one or more candidate molecules to said animal or its progeny and observing any changes in activity of said protein implicated in metabolism.


SEQUENCE LISTING (10 sequences)

SEQ ID NO: 1 - DNA, 2911 bp, Drosophila melanogaster
ttcggcacgaggttttttcgttttaaatcgcaaaaaacacaaacaaatcagtgataaataatattgcaaccagacggtagctcaagcggaactggcaaccaatgattacgaattaacaacaaacaagtgtatgtgcacttcagttattgactaactcgccacttccttttgctcgcaacaacaaaacaacgcaaaagaatattttgagcggatgtgcgtgtgtttgtgtgtgtgtgcgtgtgagcgagatatcatgcactttgtgtagaaaaataaaagcataaaaaagatgccgagcatttagataatacttaaaccgcaaacgcttttgaaacggactccagctgggtaactcgtgtggcaaactgaaccggaaccggcagaggcagcggcagcaggagcaggaggaggacctggaccagcggaactggcaaccacctggccaaatgggtggcaatagcacaggagcgaatacgagcgccttcagcgctggcggttacattgggcccacgtcggccagcagtcatcacagcctgggaacttcatcggcggcagcggcagcagcagcaggaagtgacctgatacccgcaccaattggcacgggcaacgccatgggagtgtcttcgtatgcatacggtggaaccagttgggaggagttctgcgaacgacacgccagagtggctgcctcggatttcgccaaggcgtgcatcacgtacattaatggcaatctgccgccggaggaggcgaggaacatccagcatcgcagctttgctcagaaatttgtggaatccttttcggcgcactacgacacggagttcttcaagcggaggagcactcttaaatcaggtgcgggctcgctggacttcgaggaggagcacgaggtgccaaaactgctctcaaagtctctattaagacgactatcattcaaaggactgcgcaagggcaaggccttcttccacaagaactcggatgacttggatggcagcggtggcagcggcaagcagagcaagacgaagctggccaagatcgttgtggagtgccggaaggagggcacagtgaacaacctgacgccggaaagtctggatcaaccgacgggctcccaaaagtgggagaagtgtcgacttgtactggtcaaggccgtgggtggctacatgctagagttttacacgccgcacaaggcgactaagccgcgcagtggagtcttttgtttcctcatctcggaggctcgcgaaacaacggcacttgagatgccggacaggctgaacacattcgtcctcaaggcagacaacaacatggagtatgtgattgaggcggagagcgcggaggagatgcggagttggctagccacaatacggtactgcatgcgaacgccacccactcagcagccaacgatcgagtcggatggtgttatggcgtccgccatgcaaacatcgccgacacttccgagtcccaatcccattggtgggattcagaatccgcagtaccagcagcagcgcggctcgaatggcaatctggtgggaggtggagctccgctcacctcatcgctgtctgcagacagtgctttgggccagggaggagccacttctgccagcgaactaaatgtcatcaacgaattgggcacctcaccgacctccgggccacctgacatacctgtaagaccccatcgaggtgaacagcgcctgtccgcctcaagcaacttcgatggcatcgagggcacggaaaatgatgcagatgtggcggatctgacggctgagatgagcgtgtttccctggttccacggcacactgacgcgatcagaagctgccagaatggtactccattcggatgcagccggacatggatactttttggtgcgacagagcgaaacgcgccgcggcgagttcgttctgacctttaacttccaaggacgagccaagcatttgcggctcaccatttcggagaagggtcagtgtcgggtgcagcacctgtggtttccctcgatccaggaaatgctcgaacacttccgccacaacccgataccactggaatcgggcggcacttcggatgtgactcttaccgaatgggtgcactcacacagcagactgaatgacccgacgacggcagcaaatcatgactcaggacaactcaacgatctttcgacaaatgggaatggcaatgggaacggcaatggctacgataatggtcagggttcatcgacggcatcgaatgcggcgggaggaactgcatcgggagctgctggcggtggccatccgtcgccgagacatgtgagatagccaaaattttggagtcaattcagttgcaacgaagtgattaccatgaatttgagtgttcgcctaaagacaaatgaaatcgaactgccacaggagccaacacacgtctattttccggagcaagtctatttccatttggatcccacaacgctaacagtgcacggatcaccgccaccggcccagaatttcctggaccagccacatctgcgggcttcgaacgcctcccttcaggcagctgcccaccatcaggcgggttcctccggcaaccggcatcccagcgatggcggcagcaacagcggcggagcaggaggcggatcgggatccagtggaggagccgagtgcaccggacgggccgtcgataatcagtacagcttcacctaagtccgcgcgatcgatcatcaactgcatttcgcggcttaaccagcggaactttacttttgtgccattttaatcattgtcctaaaggagaggaaaattgtgttttttttcgcatcaatgtgcgttctttgctttcttgattcacttgtttctattttagttagtacctcttttggaagacatgaagtaactgaacattattatgtatacattatatagctaaatgtgtgtgttcattttaagtacatcaatgttgtatgtat

SEQ ID NO: 2 - protein, 608 aa, Drosophila melanogaster
MGGNSTGANTSAFSAGGYIGPTSASSHHSLGTSSAAAAAAAGSDLIPAPIGTGNAMGVSSYAYGGTSWEEFCERHARVAASDFAKACITYINGNLPPEEARNIQHRSFAQKFVESFSAHYDTEFFKRRSTLKSGAGSLDFEEEHEVPKLLSKSLLRRLSFKGLRKGKAFFHKNSDDLDGSGGSGKQSKTKLAKIVVECRKEGTVNNLTPESLDQPTGSQKWEKCRLVLVKAVGGYMLEFYTPHKATKPRSGVFCFLISEARETTALEMPDRLNTFVLKADNNMEYVIEAESAEEMRSWLATIRYCMRTPPTQQPTIESDGVMASAMQTSPTLPSPNPIGGIQNPQYQQQRGSNGNLVGGGAPLTSSLSADSALGQGGATSASELNVINELGTSPTSGPPDIPVRPHRGEQRLSASSNFDGIEGTENDADVADLTAEMSVFPWFHGTLTRSEAARMVLHSDAAGHGYFLVRQSETRRGEFVLTFNFQGRAKHLRLTISEKGQCRVQHLWFPSIQEMLEHFRHNPIPLESGGTSDVTLTEWVHSHSRLNDPTTAANHDSGQLNDLSTNGNGNGNGNGYDNGQGSSTASNAAGGTASGAAGGGHPSPRHVR

SEQ ID NO: 3 - DNA, 1683 bp, Drosophila melanogaster
cgagaacagttgggcggcatcatgtttctaatagccattgccattattttggccaccattttggtgttcaagggagtgaggatattcaactacatagaccacatggctggcatcatggagatgatcccaggacccacgccataccccttcgtgggtaatctgttccagttcggtctcaagccagccgaataccccaaaaaggtcctgcaatattgtcggaaatatgacttccagggattccgctccctggtcttcctgcagtaccacatgatgctgagtgatccggctgaaattcagaacatcctgtcgagctcatcgctgctgtacaaggagcacttgtactcctttctgaggccctggctgggcgatggcctcctcaccagttccggtgcccgctggctaaagcaccagaagctctacgcccctgccttcgagcgctcggccatcgagggttacctgcgagtggtccaccggacgggcggacagttcgtccagaaactcgacgtactgtcggatacacaggaagtcttcgatgcccaggagctggtggctaagtgtaccctggatattgtgtgtgaaaacgccactgggcaggacagcagctcactgaatggagagacttcggatttgcatggagccatcaaggacttatgcgatgtggtccaggagcgcaccttcagcatcgtgaagcgtttcgacgccctcttccgcctcacctcctactacatgaagcagcgccgcgctctgtcgctcctgcgcagcgaactgaatcggattatctcgcaacggcgacaccagttggctgcggaaaacacgtgccagcagggccagccaatcaacaaacccttcctggacgtcctgctgaccgccaagctcgatgggaaagtcctcaaggagcgcgagattatcgaggaagtgtccacatttatatttacaggtcacgatcccattgccgccgccatatctttcacgctgtacaccctttcccgtcactcggagattcagcaaaaagctgccgaggaacagcgacgcatctttggcgagaacttcgcgggggaagcggacttggctcggctggatcagatgcattatctagagttgattattagggagaccctgcgcttgtacccttctgtcccactgattgctcgaacaaaccgcaatcccatcgatatcaatggcaccaaggtggccaagtgcaccacggtgatcatgtgcctcattgccatgggctacaacgaaaagtacttcgacgatccatgcacattccggccagagagattcgagaacccaactggaaacgtgggcatcgaggctttcaagagcgttccatttagtgcaggtccaaggcgctgcattgccgagaagttcgccatgtaccagatgaaggctttgctgtcccaattgctgcgccgctttgaaattctgcctgccgtggatggacttcctccgggaattaacgaccattcccgcgaggattgtgtcccacagagcgagtacgatcctgtgttgaatattcgtgtcacgcttaaatcggaaaatggtatccagattaggcttagaaagcgatgaattaacattaaaggacccttttacttgattgtattatacttcatttactagccacgatataaaataaaattgtacttttactctttttttatgaaaaaaaaaaaaaaaaaa

SEQ ID NO: 4 - protein, 516 aa, Drosophila melanogaster
MFLIAIAIILATILVFKGVRIFNYIDHMAGIMEMIPGPTPYPFVGNLFQFGLKPAEYPKKVLQYCRKYDFQGFRSLVFLQYHMMLSDPAEIQNILSSSSLLYKEHLYSFLRPWLGDGLLTSSGARWLKHQKLYAPAFERSAIEGYLRVVHRTGGQFVQKLDVLSDTQEVFDAQELVAKCTLDIVCENATGQDSSSLNGETSDLHGAIKDLCDVVQERTFSIVKRFDALFRLTSYYMKQRRALSLLRSELNRIISQRRHQLAAENTCQQGQPINKPFLDVLLTAKLDGKVLKEREIIEEVSTFIFTGHDPIAAAISFTLYTLSRHSEIQQKAAEEQRRIFGENFAGEADLARLDQMHYLELIIRETLRLYPSVPLIARTNRNPIDINGTKVAKCTTVIMCLIAMGYNEKYFDDPCTFRPERFENPTGNVGIEAFKSVPFSAGPRRCIAEKFAMYQMKALLSQLLRRFEILPAVDGLPPGINDHSREDCVPQSEYDPVLNIRVTLKSENGIQIRLRKR

SEQ ID NO: 5 - DNA, 702 bp, Drosophila melanogaster
ttcggcacgagtcgacccggctcgacccaacttaatccatttgatcgtaaagcaacctaagcagtaaacccataaccatgagcaagcctttgtccttcatctcgatggtggccgtgattttgctggccagctccacagtgaagttggcccaaggaacgctctgcagtgaaaagctcaacgaggtgctgagtatggtgtgcgaggagtataatcccgtgattccacacaagcgcgccatgcccggtgccgacagcgatctggacgccctcaatcccctgcagtttgtccaggagttcgaggaggaggataactcgatatcggaaccgctgcgaagtgccctctttcctgggagctatcttgggggtgtactcaattccctggctgaagtccggaggcgaactcgccaacggcaaggaatcgtggagaggtgctgcaaaaagtcctgtgatatgaaggctctgcgggagtactgctccgtggtcagaaattaggcctcctaatgcgaaaatcattgaccccaactgacctggtcgacgcgattatctctggatctggttccaaaccaaccatgtgcatatatactacaatcgatgttttttacagcttgttgcatgttactctttacgaatgatcgaaatggattaaatatatattctgctttaagctttggcaaacaatcgcaaaaaaaaaaaaaaaaaaaaa

SEQ ID NO: 6 - protein, 137 aa, Drosophila melanogaster
MSKPLSFISMVAVILLASSTVKLAQGTLCSEKLNEVLSMVCEEYNPVIPHKRAMPGADSDLDALNPLQFVQEFEEEDNSISEPLRSALFPGSYLGGVLNSLAEVRRRTRQRQGIVERCCKKSCDMKALREYCSVVRN

SEQ ID NO: 7 - DNA, 1757 bp, Drosophila melanogaster
ttcggcacgagaataaacataaataacgacatctgagtattctataaataatacgggcaactagcgcttcctagtccaattaaagcggcttaaaaaattagtgctgaagaattaacggttacataaatgaaagttgcgtgtgcaaaacgcctgtcgcttaatttgctgatgctgtcaaatccgtaaacctgttttggatgcccatgaactcatcaacagccaccttgtgacgtccaaaacaacaagtattaatcatagataattagacggcggaaaaatggttacggccacggcgattgcggatctgtgcatcttccttttgacctggaacgtgggcacccatacgccgcgaaatcaggatctgagctcccttttgtcccttaatggcaccacctcttgtccggataaccagctgcccgacatctatgtgatcggattccaggaggtgagcaacacaccgcaggtgctaaaaatcttcaatgacgatccgtgggtgctgaagatcgcggactctctgagcgatcaccagttcgttaaggtggactcgaagcagctgcagggaattcttataaccatgttcgcacagcacaagcacatcccgcacatgaaagaaatcgagacagaagccacgcgcacgggacttggagggctgtggggcaacaagggtgccgtgagcattcgactttccctctacggaactggcgtcgcattcgtctgctcccatctggcggcgcacgatgagaagctgaaggagcgcatcgaagactatcaccaaatcgtggacaatcacaaatacaatgcgcagggttatcgacggatattcgatcacgactttgtcttttggtttggcgatcttaacttccgtctctccggcgatatgtccgcttgggatgtccgcacggatgtggagaatcagcgatacgctgatctgctcaagctggaccagttgaatctgctccgggagaagggcaacgccttcagcttgctggaggagcagcagcccaactttgcgcccacttttaagttcgtggaaggcactaatgactacaacttgaagcggcgacccgcctggtgcgatcggattttgcatcgcgtgcagagcaacatttatccgggcattaccctgagtgccaaccagctgtcttatcagtcccacatggactacactctctccgaccacaagcccgtatcggcgacattcaactacaaggtcgaggctgccaaccagacctacaccgacgaggagctccacgaaatgacgcacggatctgcctcatctccggcgacgccaaatgttagtctatccttcgcatttgttgtttttgtagctgtgtcctatactcaactttgatttcgcttcttttgtagttatttatcgctgaactagagcaatggtagcgccgtcatgacgtcacttagttgtgaacttttaaatctctttgcatgcttttagattatatgcgtagacgagttgtagagtaattaaaagacttcctttcagaaggaacaagagaaacgaaaataatatcaagtacaatgtgatgaaatctctgctaatttagcaatcatttaagcaattctcaagtgatccatggaaataatgcagcttccgagtatatatacatacatatgttatcactcaattaagacgatattaatttaacgattgattgcattaaacaaatgtatttttgtacactaaactgttgatcgaataaaaaaaactattacctctcgaaaaaaaaaaaaaaaaaaaa

SEQ ID NO: 8 - protein, 357 aa, Drosophila melanogaster
MVTATAIADLCIFLLTWNVGTHTPRNQDLSSLLSLNGTTSCPDNQLPDIYVIGFQEVSNTPQVLKIFNDDPWVLKIADSLSDHQFVKVDSKQLQGILITMFAQHKHIPHMKEIETEATRTGLGGLWGNKGAVSIRLSLYGTGVAFVCSHLAAHDEKLKERIEDYHQIVDNHKYNAQGYRRIFDHDFVFWFGDLNFRLSGDMSAWDVRTDVENQRYADLLKLDQLNLLREKGNAFSLLEEQQPNFAPTFKFVEGTNDYNLKRRPAWCDRILHRVQSNIYPGITLSANQLSYQSHMDYTLSDHKPVSATFNYKVEAANQTYTDEELHEMTHGSASSPATPNVSLSFAFVVFVAVSYTQL

SEQ ID NO: 9 - DNA, 4175 bp, Drosophila melanogaster
ttcggcacgaggcacagctgccgccggtgcatgtcattgctgtgtgtgcgtgtgtgggtgaacttgagtgggaggcggaagatattggaaaaattcctccgcttttcgatgtcttagctttagctggacgcaaaacttaaactgcagacagtaaaggacgaaatcacattactcgactcaaaagaagacacacggaagagagataccaacaagatggccatgtccaaggtgatccgtgtgctggagaagtccattgccccctcgccgcacagcgtattactggagcatcggaacaagagcgacagcatcctgttcgagtcccatgcggtggccctgctgacccagcaggagacggatgtcatccggaagcagtacaccaaggtctgcgacgcctacggatgtctgggtgccctccaactaaacgccggcgagagcaccgtgctgttcctggtgctggtcaccggctgtgtgtccatgggcaagatcggcgacatcgagatcttccggatcacacaaaccacttttgtctcgctacaaaatgcagcgcccaacgaagacaagatcagcgaggtgcgcaagctgctcaactcgggcaccttctactttgcccacaccaatgccagtgcatcggcatccggagcgtcatcgtatcggttcgatattacgctttgcgcccagcgacgccagcaaacgcaggagacggacaaccgtttcttctggaatcgcatgatgcacatccacctgatgcgcttcggtatcgattgccagtcctggttgttgcaagccatgtgcggctccgtagaagtgcgcaccgtctacatcggtgccaaacaggcccgtgccgccatcatttcccgactgagctgcgaacgggctggcacgcgtttcaatgtccgtggtaccaacgatgagggctatgtggctaactttgtggaaaccgagcaagtgatctacgtggacggcgatgttactagttatgtgcagacgcgaggatcggtgccactcttctgggaacagccaggcgtccaggttggctcgcacaaggtgaagctatcacgaggattcgagacatcggccgccgcctttgatcgacacatgagcatgatgaggcaacgttacggctatcagacggtggtgaatctgctaggcagctcccttgttggcagcaaggagggcgaggccatgctgagtaatgagttccagcgtcatcacggcatgtcagcccacaaggatgtgccgcatgtggtgtttgactatcatcaggagtgtcgcggcggcaatttctcggcgctggccaagctcaaggaacggatcgtagcctgcggtgctaactacggcgtcttccacgcgtccaatggtcaggtgttgcgcgagcagttcggtgttgtgcgcacgaattgcctagactgtttggacaggacaaactgtgtgcagacgtatctcgggcttgacacgctcggtatccagctagaggctttgaaaatgggcggcaagcagcagaatatttcgcggtttgaggagatcttccgacagatgtggatcaacaacggaaatgaggtcagcaagatctacgccggcaccggggccatccaggggggatcaaaactaatggatggtgcgcgatctgcagcaagaacaattcaaaataacctactggacaactcaaagcaggaagccattgatgtcctgctagtgggctccacgcttagctcggagcttgcggatcgggctcgcatcctactgccctccaatatgttgcatgcacctaccactgtgttgagagagctatgcaagcgctacactgaatatgtgcgtcctcgaatggcacgtgtagccgtgggtacctataacgtcaacggcggcaagcacttccgcagcattgtgttcaaggattcgctggccgattggctgctcgactgccatgcccttgcccgctccaaggcgcttgtagatgtgaacaatccgtcggagaacgtcgatcatccggtggatatctacgccattggattcgaggagattgtggatctgaatgcttccaacataatggcggccagcaccgacaatgccaagttgtgggccgaggagctgcagaaaacgatctcgcgggacaatgactacgtgctgctcacataccagcaactggtgggcgtgtgcctatacatctacatccgaccggagcacgcgccgcacatccgggacgtggccatcgactgtgttaagacaggattgggtggtgccactgggaataagggtgcctgtgccattcgatttgtgcttcatggtacttccatgtgcttcgtgtgtgcccactttgcagccggacagtcacaggtggctgaaaggaacgctgactacgcggaaatcacccggaagctggccttcccgatgggcaggacgctaaaatcacacgactgggtgttttggtgcggcgacttcaactatcgcatcgacatggagaaggacgaattaaaggagtgcgtacgtaatggagatctctcaaccgtcctcgagttcgatcaattgcgcaaggagcaggaggctggcaatgtgtttggcgaattcctcgagggagagatcactttcgacccgacgtacaagtatgatttgttcagcgacgactacgacacctcggagaaacagcgagcgcccgcctggacagatcgggttctctggcgtcgcaggaaggcgctggccgagggcgactttgctgcctcagcctggaatcccggcaaattgattcactacggtcgttcggagctaaagcagagtgatcatagacccgtgatcgctattattgatgctgagataatggagatcgatcagcagcggagacgtgctgtattcgagcaggttatccgagatctgggtccgccggactccacgattgtggtacatgtcctggagtcttcagcaactggagatgaggatggaccaaccatatacgacgagaatgtgatgtcagccctgattaccgagctatccaagatgggagaggtcactttggtgcgctatgtggaggacaccatgtgggtcacatttcgggatggagaatcggctttaaatgcgtcttctaagaagagtatccaagtatgcggattagatcttatcctggagctgaagtcaaaggactggcagcatctggtggacagcgagatagagctatgcaccacgaacaccataccgctgtgcgctaatcctgtagagcatgcacaactcctgcaggccattacgccggagttgccccagcggcccaagcagccgcccacacgtcccccagcccgtcccccaatgcctatgtcgccaaagaactcaccacgccacctgccccacgtcggggtcatcagcattgtgcccaagccggcaaagccaccgatgccaccgcaaccgcaatctcaacctctaattccgtcgccgcttcagccgcaggtggcgccgcctcgtccgccggcaatggacaccacgccatcctccaaatcgcaatcgccaacggaacttgtatccgctagttcttcgacgtcctcttcgggaaagacttcgcccaccacccataccaaataggagcggcaatgctccaccgctgcccacgcgacccgccaacaactgagctgcaggctacgcacacgggctcgctgtcttgcactccgattgcattccgaatcgctgtatttaatgttatatatactcgaatatattgggccactctggaggagaactcctggcaatatccgcacttcgcatggaatctatgttactacctgtttgtttgtttcgatagtttctagccggaatcaaactgaatttaaagtaaacaaataagttccacatagctaaagcccaaactaccccactccggtactctcttttttttttttttacttactggccagacgtactcaacccaattgcaccggatttgccgagggcgaagaatgaatataatgaagtatatgggcgacaaagtattacactaatctgcataaatgtaatgtttaaataaattatagccgtactagtcaaattatgaagtaaagttataaaaactgaagaagcaatagaactctgtaaaagattccgattcgagcgacacacccaaacacatgtatccattgttattgaacaactattgagataaattacatattattccacattgttaaaaaaaaaaaaaaaaaaaa

SEQ ID NO: 10 - protein, 1114 aa, Drosophila melanogaster
MAMSKVIRVLEKSIAPSPHSVLLEHRNKSDSILFESHAVALLTQQETDVIRKQYTKVCDAYGCLGALQLNAGESTVLFLVLVTGCVSMGKIGDIEIFRITQTTFVSLQNAAPNEDKISEVRKLLNSGTFYFAHTNASASASGASSYRFDITLCAQRRQQTQETDNRFFWNRMMHIHLMRFGIDCQSWLLQAMCGSVEVRTVYIGAKQARAAIISRLSCERAGTRFNVRGTNDEGYVANFVETEQVIYVDGDVTSYVQTRGSVPLFWEQPGVQVGSHKVKLSRGFETSAAAFDRHMSMMRQRYGYQTVVNLLGSSLVGSKEGEAMLSNEFQRHHGMSAHKDVPHVVFDYHQECRGGNFSALAKLKERIVACGANYGVFHASNGQVLREQFGVVRTNCLDCLDRTNCVQTYLGLDTLGIQLEALKMGGKQQNISRFEEIFRQMWINNGNEVSKIYAGTGAIQGGSKLMDGARSAARTIQNNLLDNSKQEAIDVLLVGSTLSSELADRARILLPSNMLHAPTTVLRELCKRYTEYVRPRMARVAVGTYNVNGGKHFRSIVFKDSLADWLLDCHALARSKALVDVNNPSENVDHPVDIYAIGFEEIVDLNASNIMAASTDNAKLWAEELQKTISRDNDYVLLTYQQLVGVCLYIYIRPEHAPHIRDVAIDCVKTGLGGATGNKGACAIRFVLHGTSMCFVCAHFAAGQSQVAERNADYAEITRKLAFPMGRTLKSHDWVFWCGDFNYRIDMEKDELKECVRNGDLSTVLEFDQLRKEQEAGNVFGEFLEGEITFDPTYKYDLFSDDYDTSEKQRAPAWTDRVLWRRRKALAEGDFAASAWNPGKLIHYGRSELKQSDHRPVIAIIDAEIMEIDQQRRRAVFEQVIRDLGPPDSTIVVHVLESSATGDEDGPTIYDENVMSALITELSKMGEVTLVRYVEDTMWVTFRDGESALNASSKKSIQVCGLDLILELKSKDWQHLVDSEIELCTTNTIPLCANPVEHAQLLQAITPELPQRPKQPPTRPPARPPMPMSPKNSPRHLPHVGVISIVPKPAKPPMPPQPQSQPLIPSPLQPQVAPPRPPAMDTTPSSKSQSPTELVSASSSTSSSGKTSPTTHTK